(19) United States
(12) Patent Application Publication
(10) Pub. No.: US 2007/0117182 A1
(43) Pub. Date: May 24, 2007

(54) Nucleic acids conferring transcriptional responsiveness on the RANKL gene promoter and uses thereof

(75) Inventors: John W. Pike, Madison, WI (US); Nirupama K. Shevde, Madison, WI (US)

Correspondence Address: GODFREY & KAHN, S.C., 780 N. WATER STREET, MILWAUKEE, WI, 53202, US

(73) Assignee: Wisconsin Alumni Research Foundation

(21) Appl. No.: 11/283,599

(22) Filed: Nov. 18, 2005

(51) Int. Cl. C12P 21/06; C07K 14/705; C12N 9/64; C07H 21/04; C12N 5/06

(52) U.S. Cl. 435/69.1; 536/23.2; 435/320.1; 435/325; 435/348; 435/349; 435/226; 530/350

(57) ABSTRACT

The present invention provides isolated nucleic acids containing functional polynucleotide sequences from the upstream region of the RANKL gene useful in conferring transcriptional responsiveness (e.g., vitamin D₃ receptor complex responsiveness) on associated promoters and methods of using same to identify chemical entities capable of affecting transcriptional activity of the RANKL gene.

### STATEMENT REGARDING FEDERALLY SPONSORED RESEARCH

This invention was made with United States government support awarded by the National Institutes of Health—Grant No. DK056059. The United States has certain rights in this invention.

### FIELD OF THE INVENTION

The present invention relates to the process of osteoclastogenesis. More particularly, the present invention relates to the receptor activator of NF-kB ligand (RANKL) and DNA regions that confer transcriptional responsiveness on the RANKL gene promoter.

### BACKGROUND OF THE INVENTION

The bone forming osteoblast and the bone resorbing osteoclast comprise the primary cells that regulate skeletal homeostasis. These cells act in concert to direct the continual remodeling of bone that occurs during adulthood. The process of bone renewal is initiated by bone lining osteoblasts which respond to locally generated mechanical or chemical signals and elaborate regulatory factors capable of stimulating both the activity of preexisting osteoclasts and promoting the formation of new osteoclasts essential to the bone resorption process. As signals from the bone lining cells decrease, the bone resorbing phase of the process yields to the recruitment of new osteoblasts, the formation of new bone matrix and re-mineralization, all of which restore new bone and complete the bone remodeling cycle.

The primary osteoclastogenic signal produced by stromal cell-derived bone lining cells is receptor activator of NF-kB ligand (RANKL), a TNF-like factor that is essential to the formation, activation and survival of bone resorbing osteoclasts. Indeed, RANKL-null mice prepared using homologous recombination do not produce osteoclasts, cannot resorb bone and as a consequence manifest osteopetrosis resulting in morbidity. Levels of RANKL produced by bone lining osteoblasts and stromal cells maintain an appropriate level of osteoclast formation necessary for normal bone turnover and skeletal integrity. RANKL expression also serves a more global physiological function, however, to liberate both calcium and phosphorus from the skeleton and to contribute to the maintenance of blood mineral levels within precise limits during times in which dietary calcium is insufficient. This liberation provides a backup system to ensure appropriate extracellular calcium and phosphorus homeostasis. Since this feature of all vertebrates is under the purview of the calcium regulating hormones 1,25(OH)₂D₃, PTH and phosphatonin (FGF23), it is therefore not surprising that these hormones represent primary regulators of RANKL expression. Unfortunately, increases in blood levels of these hormones either as a result of a pathological state or as a result of therapeutic treatment exert profound effects on RANKL production and, if left unchecked, result in hypercalcemia, systemic bone loss and potentially osteoporosis. This catabolic effect of 1,25(OH)₂D₃, which is provoked through a normal physiologic response, has limited the use of 1,25(OH)₂D₃ and analogs for diseases such as psoriasis, diabetes, lupus and multiple sclerosis as well as cancer, indications for which they have shown possible therapeutic efficacy.

RANKL is expressed in response to both systemically as well as locally produced cytokines such as IL-1, IL-6, and TNFalpha as well as a variety of additional immunoregulatory molecules. While the bone resorptive component of these cytokine-induced responses plays an important physiological role in nature in its initial phase, the bone resorption can lead eventually to skeletal disease pathology. Examples of skeletal disease pathologies induced by cytokines include the loss of bone associated with local inflammation, arthritis, wear debris and other types of osteolysis, and the bone loss associated with tumor growth.

The above-described observations have prompted pharmaceutical development of inhibitors or antagonists of RANKL such as the RANKL decoy receptor osteoprotegerin (OPG) (Morony S, et al., J Bone Miner Res. 14:1478-1485 (1999)), a soluble version of the receptor for RANKL (RANK) (Wittrant Y, et al., Biochim Biophys Acta. 1704:49-572 (2004)), and several neutralizing antibodies capable of controlling bone loss. The nature of the actions of these molecules highlights the current focus of blocking RANKL biological activity rather that suppressing its expression. A significant limitation of this approach is that inhibition of RANKL activity requires large molecules such as proteins or antibodies. The delivery of these molecules is possible but highly problematic and generally undesirable. No efforts appear currently in progress to identify transcriptional inhibitors of RANKL expression, although this is a promising avenue for drug discovery. Whether the current molecules will be effective in the treatment of diseases of RANKL overexpression remains to be determined.

Inhibitors of RANKL expression are preferably small molecules, which can be delivered orally and whose potency, efficacy and selectivity can be enhanced or modulated via deliberate chemical modification. These small molecules provide a significant advantage over the current protein or antibody based therapeutics. In order to identify transcriptional inhibitors of RANKL expression, a molecular understanding of RANKL gene expression from osteoblasts is necessary. However, traditional approaches for characterizing transcriptional regulation have failed to identify regulatory regions within the proximal RANKL promoter capable of such regulation. Such traditional approaches typically entail an initial cloning of the proximal promoter region followed by subsequent fusion of these regions to a reporter gene such as firefly luciferase. The activity and responsiveness of these DNA constructs is assessed following transfection into host mammalian cells. Interestingly, this approach has not been successful in identifying the regulatory regions responsible for conferring transcriptional responsiveness on the RANKL promoters. Although the RANKL promoter from both mouse and human have been identified and reporter constructs prepared, none have exhibited response to 1,25(OH)₂D₃, PTH or other known activators of RANKL when introduced into target osteoblasts.

It can therefore be appreciated that gaining an understanding of how the RANKL gene is regulated in response to either 1,25(OH)₂D₃ or other hormonal or cytokine regulators and identifying the genetic sequences responsible for this control are important goals. Such an understanding would open routes for a wide variety of practical applications including, but not limited to, the establishment of high throughput screens capable of identifying selective small molecule inhibitors of RANKL gene transcription.

### SUMMARY OF THE INVENTION

In a first embodiment, the present invention provides an isolated nucleic acid containing polynucleotide sequence from the upstream of the RANKL gene that is capable of conferring transcriptional responsiveness on a RANKL gene promoter. Isolated nucleic acids according to the invention include a RANKL upstream polynucleotide sequence forth in SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a polynucleotide sequence having substantial sequence homology thereto that is capable of conferring transcriptional responsiveness on an operatively linked RANKL gene promoter.

In certain preferred embodiments, a promoter is included in the isolated nucleic acid that is transcriptionally responsive to the RANKL upstream regions described and claimed herein. Suitable promoters include, for example, a minimal RANKL gene promoter or a Herpes simplex virus thymidine kinase promoter. Certain other nucleic acids further include a reporter gene operatively linked for transcription by the promoter. A wide variety of reporter genes may be included in the nucleic acid such as, for example, luciferase, chloramphenicol, acetyl transferase, beta-lactamase, green fluorescent protein, or beta-galactosidase genes.

In another embodiment, the invention is directed to a host cell that contains an isolated nucleic acid including RANKL upstream regions that is capable of conferring transcriptional responsiveness on an operatively linked RANKL gene promoter. In certain embodiments, the host cell endogenously expresses a steroid/thyroid hormone receptor or other transcription factor capable of interacting with the RANKL upstream region and regulating RANKL gene expression (e.g., the vitamin D₃ receptor).

In yet another embodiment, the invention provides a method for identifying a chemical entity capable of altering RANKL gene transcriptional activity. Such a method includes steps of: (a) providing a host cell containing a reporter gene operatively linked to a promoter that is transcriptionally responsive to a RANKL upstream region also present in the host cell; (b) exposing the host cell to a chemical entity; and (c) measuring and comparing reporter gene expression to that of a control cell that is not exposed to the chemical entity wherein a higher or lower expression level than that of a control cell indicates that the chemical entity is capable of altering RANKL gene transcriptional activity.

The invention is also directed to a method for identifying a chemical entity having reduced hypercalcemic activity. Such a method includes steps of: (a) providing a host cell containing a reporter gene operatively linked to a promoter that is transcriptionally responsive to a RANKL upstream region also present in the host cell; (b) exposing the cell to a chemical entity; and (c) measuring and comparing reporter gene expression to that of a control cell treated with a known hypercalcemic agent wherein a lower expression level than that of the control cell indicates that the chemical entity possesses reduced hypercalcemic activity. In a preferred embodiment, the known hypercalcemic agent is 1,25(OH)₂D₃ and the chemical entity is a vitamin D analog.

Yet another embodiment of the invention is directed to a method for the controlled expression of a gene. Such a method includes the steps of: (a) providing a host cell containing a RANKL upstream region further containing a gene operatively linked for transcription to a promoter that is transcriptionally responsive to a RANKL upstream region also contained within the host cell ; and (b) culturing the host cell under conditions to express the gene. In certain embodiments, the host cell endogenously expresses a steroid/thyroid hormone receptor or other transcription factor capable of interacting with the RANKL upstream region and regulating RANKL gene expression (e.g., the vitamin D₃ receptor).

As can be appreciated, it is one object of the present invention to provide methods for screening chemical entities and identifying same that can alter the abilities of RANKL upstream control elements described and claimed herein to confer transcriptional responsiveness on associated promoters. This invention provides the advantage over prior technologies in that embodiments of the invention utilize or are based on the native response elements from the RANKL gene, as recently discovered and characterized by the present inventors. Other objects, features and advantages of the present invention will become apparent after review of the specification, claims and drawings.

### BRIEF DESCRIPTION OF THE DRAWINGS

FIG. 1. Regulation of osteoclastogenesis by calciotropic hormones and other factors via RANKL and the stromal/osteoblast.

FIG. 2. Induction of RANKL in ST2 cells by hormones and cytokines. ST2 cells were treated with the agents indicated for periods up to 24 hours. Total RNA was isolated and subjected to RT-PCR using a primer set to mouse RANKL and beta-actin. Positive vitamin D target control genes include Cyp24 and osteopontin (OPN).

FIG. 3. ChIP/chip analysis of the RANKL gene locus. A) ChIP: ST2 cells were treated with either vehicle or 1,25(OH)₂D₃ (10⁻⁷ M) and after 6 hours subjected to ChIP using antibodies to VDR, RXR and IgG. Precipitated DNA was analyzed for the presence of either the Cyp24 or the OPN promoter. B) Arrangement of the mouse RANKL gene locus and position of flanking genes. RANKL gene is transcribed on the reverse strand (right to left). C) DNA chip: Precipitated DNA was linearly amplified and then subjected to chip analysis using tiled oligonucleotide arrays containing 50-mers spanning the RANKL gene locus as described in the text. Hybridization data derived from the arrays are indicated in log₂ scale across the locus with nucleotide positions on chromosome 14 indicated on the X axis. The first set of two arrays provide linear data comparing IgG (±1,25(OH)₂D₃) and VDR (±1,25(OH)₂D₃) over the RANKL upstream regions. The second set of three arrays provides an expanded view of hybridization data comparing VDR (±1,25(OH)₂D₃), VDR (+1,25(OH)₂D₃ vs input DNA) and RXR (+1,25(OH)₂D₃ vs input DNA). VDR/RXR binding regions D1-D5 are tracked by the shading.

FIG. 4. Confirmation of ChIP/chip data using ChIP. A) Linear arrangement of RANKL locus re-oriented 5′ to 3′ (left to right) indicating the positions of D5 to D1, P2, and P1/TSS. Designation of primer sets used for detection in ChIP is indicated. B) ST2 cells were treated with either vehicle or 1,25(OH)₂D₃ and then subjected to ChIP using antibodies to VDR, RXR or IgG. Precipitated DNA was evaluated using the primer sets identified in A.

FIG. 5. ChIP analysis of VDR/RXR, GR, C/EBPβ and RNA pol II binding in response to 1,25(OH) 2D3, Dex or the combination. ST2 cells were treated with the above hormones and after 6 hours subjected to ChIP analysis using antibodies to VDR, RXR, GR, C/EBP, β, RNA pol II, and IgG. Precipitated DNA was evaluated for enrichment of the indicated regions of the RANKL gene. As can be seen, C/EBPP binds extensively across the regions. In contrast, the VDR binds only to the five upstream regions (D1-D5) of the RANKL gene (D4 binding is again the weakest), but not to intervening regions or to those proximal to the promoter (where VDREs have been suggested to reside). GR binding is dependent upon glucocorticoids, and is limited to the D5, D3 and D1 regions of the gene. Finally, RNA pol II is recruited to each of the D1-D5 sites as well as the TSS as a function of 1,25(OH)₂D₃.

FIG. 6. Transcriptional activity of the D5 regions of the RANKL gene in the context of the TK promoter. Sequence surrounding the D5 region (1072 bp) was cloned into the pTK-luciferase vector and termed pTK-RL(D5). A) ST2 cells were transfected with pTK-luc or pTK-RL(D5) and a VDR expression vector and then treated with either vehicle, 1,25(OH)₂D₃, Dex or both. Cells were harvested after 24 hours and luciferase activity assessed. B) ST2 cells were transfected as in A above but without the VDR expression vector and evaluated as in A. C) COS-7 cells were transfected and evaluated as in A above. Luciferase was normalized using beta-gal activity. Data represent the mean of triplicate determinations (SEM). The pTK-luc control vector was unresponsive to any of the inducers (see FIG. 9). *, p<0.05 vs no treatment control.

FIG. 7. Mapping the VDRE(s) in the RL-D5 region of the RANKL gene. A) The D5 fragment was subjected to deletion analysis to produce the subfragments indicated (5′ and 3′ boundaries and sizes shown). B) Fragments were cloned into the pTK vector and evaluated for activity by transfection in ST2 cells in response to hormones. Data represent the mean of triplicate determinations (SEM). As indicated, both 1,25(OH)₂D₃ response and Dex potentiation was lost in the third fragment RL-D5-2. C) Sequence and species conservation of the VDRE(s) identified in B as well as in silico derived CREs located immediately upstream. *, p<0.05 vs no treatment control.

FIG. 8. The mouse RANKL VDRE is inducible by 1,25(OH)₂D₃. The mouse RANKL VDRE (sequence seen in FIG. 7) was cloned into the TK promoter, transfected into ST2 cells and evaluated for transcriptional regulation by 1,25(OH)₂D₃. A 10 to 15-fold induction mediated by the RL VDRE is observed. A similar induction is observed in the context of the RL minimal promoter. Data are the average of triplicate determinations, SEM.

FIG. 9. The mouse RANKL D5 region is active in the context of the mouse RANKL gene minimal promoter. Mouse RL D5 was inserted upstream of a mouse RANKL gene promoter fragment (−100 to +65) in pGL3 and its inducible activity assessed in ST2 cells. Insertion of RL D5 resulted in activity similar to that seen in the context of the TK promoter, while the RL minimal promoter was inactive. Data are the average of triplicate determinations, SEM.

FIG. 10. ChIP analysis of transcription factor binding to RL D5, the TSS and intervening regions of the RANKL locus in response to 1,25(OH)₂D₃, forskolin and oncostatin M (OSM). ST2 cells were treated for 6 hr with the indicated inducing agents and then subjected to ChIP analysis using antibodies to the indicated factors. Precipitated DNA was analyzed by PCR for enrichment of IS7, D5, IS6 and the TSS of the RANKL gene locus. See text for details of results.

FIG. 11. Transcriptional activity of the mouse RL-CCL region of the RANKL gene in response to vehicle, 1,25(OH)₂D₃, Dex, 1,25(OH)₂D₃ and Dex, forskolin (10 7M), PGE₂ (10⁻⁷ M) and OSM (20 ng/ml). A) Coordinates of the RL-D5 and the RL-D5b regions in the mouse RANKL upstream region. ST2 cells were transfected with either B) pTK-luc or pTK-mRL-D5 or C) pTK-luc and pTK-mRL-D5b and then treated with the indicated inducers. Cells were harvested after 24 hours and evaluated for luciferase activity. Luciferase was normalized using β-gal activity. Data represent the mean of triplicate determinations (SEM). *, p<0.05 vs no treatment control. **, p<0.05 vs no treatment and 1,25(OH)₂D₃ treated controls. As can be seen, the mRL-D5 region is responsive to 1,25(OH)₂D₃, 1,25(OH)₂D₃ and dex, and OSM, but not forskolin. The mRL-D5b region is not responsive to the above agents, but is responsive to forskolin. Note that the 10-fold increase in basal activity inherent to the mRL-D5b region relative to the mRLD5 region, perhaps suggestive of additional regulatory components within the upstream mRLD5b region.

FIG. 12. Transcriptional activities of the D1-D4 regions of the RANKL gene in the context of the TK promoter were analyzed and corresponding data is shown in FIG. 12. The D1-D4 regions of the RANKL gene were cloned into the pTK vector and termed PTK-RL(D1)-(D4). ST2 cells were transfected with pTK-luc or the pTK-RL(D1-D4) constructs and a VDR expression vector and then treated with either vehicle, 1,25(OH)₂D₃, Dex or both. Cells were harvested after 24 hours and luciferase activity assessed. Luciferase was normalized using beta-gal activity. Data in FIG. 12 represent the mean of triplicate determinations (SEM).

FIG. 13. Sequence conservation among species across the D1-D5 regulatory regions of the RANKL gene. Conservation in the TSS region as well as the D1-D5 regions of the RANKL gene in mouse, rat, human, and canine are indicated. Position and lack of conservation at intervening regions IS2, -5, and -6 are also shown in the figure.

FIG. 14. The highly conserved RL D5 region found in the human gene exhibits transcriptional activity in MG63 cells similar to that seen with mouse RL D5 in ST2 cells. A) RANKL is induced by 1,25(OH)₂D₃ and Dex in MG-63 cells. Cells were treated with the indicated inducers for the periods identified and isolated RNA subjected to PCR analysis for Cyp24, RANKL and b-actin. B) 1,25(OH)₂D₃ induces VDR binding to the conserved D5 region of the human RANKL gene in MG-63 cells. Cells were stimulated with 1,25(OH)₂D₃ and/or Dex for 6 hrs and then subjected to ChIP analysis. Two different primer sets to the human D5 region were used to assess VDR binding. VDR binding was also observed at D3-D1 but not in intervening regions or at the TSS. C) The human D5 region is transcriptionally active in MG-63 cells. Cloned mouse and human D5 regions were transfected into MG-63 cells and treated with either 1,25(OH)₂D₃ and/or Dex. Luciferase activity was analysed 24 hours later. Both mouse (mRLD5) and human D5 (hRLD5) exhibit similar responses to 1,25(OH)₂D₃ and Dex. Data are the average of triplicate determinations, SEM.

FIG. 15. Mutagenesis of individual CREs in the mouse RL D5b regions compromises forskolin response. Each of the putative CREs in mouse RL D5b were mutated by altering three bp and then evaluated in ST2 cells. Forskolin inducibility was compromised in both mutated constructs compared to the unmodified mouse RL D5b construct. Results represent the average of triplicate determinations, SEM.

### DETAILED DESCRIPTION OF THE INVENTION

### I. IN GENERAL

Before the present materials and methods are described, it is understood that this invention is not limited to the particular methodology, protocols, materials, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No: 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

### DEFINITIONS

“Host cell” is a cell which has been transformed or transfected, or is capable of transformation or transfection by an exogenous polynucleotide sequence. Certain preferred host cells according to the inventors endogenously express a steroid/thyroid hormone receptor or other transcription factor capable of regulating RANKL gene expression including factors presently known and those yet to be discovered.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215: 403-410 (1990). The BLAST X program is publicly available from NCBI and other sources (BLAST Manual, Altschul, S., et al, NCBI NLM NIH Bethesda, Md. 20894; Altschul, S., et al., J. Mol. Biol. 215: 403-410 (1990). The well known Smith Waterman algorithm may also be used to determine identity. The term “substantial sequence homology” refers to DNA or RNA sequences which have de minimus sequence variations from, and retain the same functions as, the actual sequences disclosed and claimed herein.

“Isolated” or “purified” or “isolated and purified” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Moreover, a polynucleotide or polypeptide that is introduced into an organism by transformation, genetic manipulation or by any other recombinant method is “isolated” even if it is still present in said organism, which organism may be living or non-living.

“Polynucleotide(s)” generally refers to any polyribonucleotide or polydeoxribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

The term “isolated nucleic acid” used in the specification and claims means a nucleic acid isolated from its natural environment or prepared using synthetic methods such as those known to one of ordinary skill in the art. Complete purification is not required in either case. The nucleic acids of the invention can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the invention in the manner disclosed herein. The nucleic acid is preferably at least about 85% pure, more preferably at least about 95% pure and most preferably at least about 99% pure.

Further, an isolated nucleic acid has a structure that is not identical to that of any naturally occurring nucleic acid or to that of any fragment of a naturally occurring genomic nucleic acid spanning more than three separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of clones, e.g., as those occurring in a DNA library such as a cDNA or genomic DNA library.

An isolated nucleic acid can be modified or unmodified DNA or RNA, whether fully or partially single-stranded or double-stranded or even triple-stranded. A nucleic acid can be chemically or enzymatically modified and can include so-called non-standard bases such as inosine, as described in a preceding definition.

The terms “transcriptional control region” or “transcriptional control element” refer to a DNA segment comprising one or more similar or different response elements capable of being operatively linked to a promoter to confer, via binding or otherwise interacting with a receptor or other transcription factor or a combination of dissimilar factors, responsiveness to transcriptional activity of the promoter.

The term “operatively linked” means that the linkage (e.g., DNA segment) between the DNA segments so linked is such that the described effect of one of the linked segments on the other is capable of occurring. “Linked” shall refer to physically adjoined segments and, more broadly, to segments which are spatially contained relative to each other such that the described effect is capable of occurring (e.g., DNA segments may be present on two separate plasmids but contained within a cell such that the described effect is nonetheless achieved). Effecting operable linkages for the various purposes stated herein is well within the skill of those of ordinary skill in the art, particularly with the teaching of the instant specification.

The term “operative to confer responsiveness to an associated promoter in the presence of a steroid/thyroid receptor or other transcription factor” means that a nucleic acid of the present invention is capable of being situated with a promoter such that, upon binding of, e.g. a steroid/thyroid receptor, the promoter is conferred with responsiveness such that a measurable change in transcriptional activity, preferably an increase in transcriptional activity, occurs at the promoter. Likewise, the term “operative to confer responsiveness to an associated promoter in the presence of a steroid/thyroid receptor” means that a nucleic acid of the present invention is capable of being situated with a promoter such that, upon binding of the receptor with cognate ligand, the promoter is conferred with responsiveness such that a measurable change in transcriptional activity, preferably an increase in transcriptional activity, occurs at the promoter.

The terms “promoter being naturally unresponsive to ligand” or “not normally subject to transcriptional activation and/or repression” mean that ligand does not enhance transcription from the promoter to an observable extent in a cell (e.g., a mammalian cell) unless a response element of the invention is spliced or inserted (upstream of the promoter) relative to the direction of transcription therefrom, by recombinant DNA or genetic engineering methods, into a DNA segment comprising the promoter, and linked to the promoter in a manner which makes transcriptional activity from the promoter operatively responsive to a steroid/thyroid hormone receptor and cognate ligand. The term “ligand” used in this context includes, but is not limited to, steroid hormone regulators such as the vitamin D₃ receptor ligand, 1,25(OH)₂D₃. The term “promoter which is not normally subject to transcriptional activation and/or repression by a vitamin D₃ receptor complex” means that vitamin D₃ receptor complex does not enhance transcription from the promoter to an observable extent in a cell (e.g., a mammalian cell) unless a response element of the invention is spliced or inserted (upstream of the promoter) relative to the direction of transcription therefrom, by recombinant DNA or genetic engineering methods, into a DNA segment comprising the promoter, and linked or situated to the promoter in a manner which makes transcriptional activity from the promoter operatively responsive to vitamin D₃ receptor complex. “Vitamin D₃ receptor complex” shall refer to the vitamin D₃ receptor in association with its cognate ligand.

The term “steroid/thyroid receptors” refers to hormone binding proteins that operate as ligand-dependent transcription factors, including identified members of the steroid/thyroid superfamily of receptors for which specific ligands have not yet been identified (referred to hereinafter as “orphan receptors”). Each such protein has the intrinsic ability to bind to a specific DNA sequence in the promoter of a target gene. Following binding, the transcriptional activity of the gene is modulated by the presence or absence of the cognate ligand. The term “vitamin D₃ receptor” refers to a hormone binding protein from any source, including but not limited to mammalian sources that operates as a vitamin D₃-dependent transcription factor. Vitamin D3 receptor has the intrinsic ability to bind to a specific vitamin D₃ receptor response element in a promoter of a target gene and, upon binding, the transcriptional activity of the promoter is modulated by the presence or absence of the vitamin D₃ ligand (i.e., the vitamin D₃ receptor complex).

The term “transcription factors” shall refer in general to transcription factors having the intrinsic ability to bind a specific DNA sequence and modulate the transcriptional activity of an associated target gene. As used in the context of RANKL gene upstream regions, the term “transcription factors” shall refer to factors from any source operating in combination with the present RANKL upstream regions to confer transcriptional responsiveness on the RANKL gene promoter. Such transcription factors include, for example, steroid/thyroid receptors, glucocorticoid receptors and C/EBPbeta.

The term “suitable or appropriate or corresponding ligand” in reference to hormone receptors of the steroid/thyroid superfamily refers to the specific ligand(s) which, in combination with its cognate receptor, is effective to transcriptionally activate the response element to which the cognate receptor binds (i.e., vitamin D₃ /vitamin D₃ receptor/VDRE).

The term “reporter gene” refers to any gene of interest where the transcription of the gene, translation of the gene product, and/or activity of the gene product can be measured. Polymerase chain reaction (PCR) may be used to measure the transcription of the reporter gene. Additionally, a detectably labeled probe specific to the reporter gene could be used to quantify the amount of reporter gene transcribed. Translation of the reporter gene may be done through use of an ELISA using an antibody specific to the reporter gene and a secondary antibody that recognizes the initial antibody. If the reporter gene is an enzyme, the activity of the enzyme may be measured using detectable substrates for the enzyme activity.

The nucleotides which occur in the various nucleotide sequences appearing herein have their usual single-letter designations (A, G, T, C or U) used routinely in the art. In the present specification and claims, references to Greek letters may either be written out as alpha, beta, etc. or the corresponding Greek letter symbols (e.g., α, β, etc.) may sometimes be used.

### II. THE INVENTION

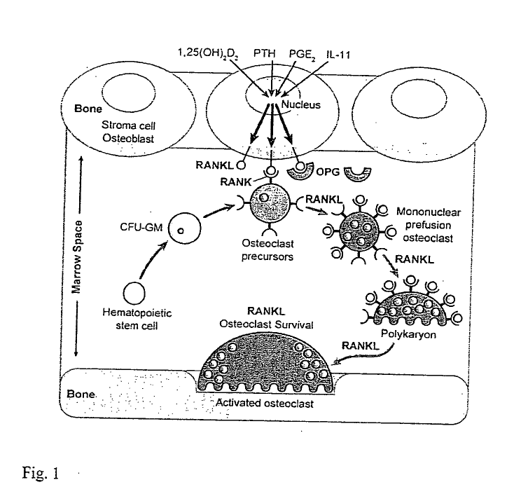

Skeletal remodeling in adults occurs through the coupled actions of bone-forming osteoblasts and bone-resorbing osteoclasts. In contrast to stromal-derived osteoblasts, osteoclasts are hematopoietic in origin and are terminally differentiated, multinucleated cells of the monocyte-macrophage lineage. The process of osteoclastogenesis, as seen in FIG. 1, is exceedingly complex and orchestrated in a highly temporal fashion through the actions of a number of hematopoietic growth factors, regulatory components and cytokines. These factors include GM-CSF, M-CSF, IL-1beta, IL-3, IL-6, IL-11, TNFalpha and receptor activator of NF-kB ligand (RANKL) as well as a variety of co-stimulatory molecules, some of which have yet to be identified. The vast majority of these regulatory factors are produced and secreted by support cells, including those from both stromal as well as hematopoietic sources. While many play a role in the normal physiological process of bone remodeling, their aberrant secretion, often in response to inflammatory or other stimuli, can lead to either focal or systemic pathological bone resorption.

Although many factors are participants in the process, the molecule that is now considered to be both necessary and sufficient for osteoclastogenesis both in vivo and in vitro is RANKL. RANKL is a recently discovered TNF-like factor that is produced largely as a membrane-associated protein by both stromal cells and osteoblasts. RANKL is actively involved not only in the differentiation process, but in the activity and survival of osteoclasts as well. The interaction of RANKL with receptor activator of NF-kB (RANK) which is expressed on the surface of osteoclast precursors triggers a number of signaling cascades including those of the IKK/IKbeta/NF-kB, the MAPKs, the SRC and the P13K/AKT pathways. Understanding of the activation of these pathways by RANKL is now significantly advanced. Importantly, activation of these pathways culminates in the stimulation of transcription factors such as c-jun and c-fos, NF-kB, the induction of NFATc1, a key member of the NFAT transcription factor family and additional regulators as well. The temporal and collective activation of these regulatory molecules initiates growth arrest and promotes osteoclast differentiation, fusion, activation and survival. The gene targets for these transcription factors include tartrate-resistant acid phosphatase (TRAP), MMP-9, cathepsin K (CathK) and the calcitonin receptor (CTR). The evidence that supports the essentiality of both RANKL and its receptor in osteoclast formation derives from the phenotypes of both RANKL- and RANK-null mice; neither of these mouse strains is capable of producing osteoclasts in vivo. Indeed, osteoclasts are not produced and bone resorption does not occur in the absence of RANKL (RANKL-null mice). Thus, animals that do not express this regulatory molecule become osteopetrotic and eventually die.

The integral role of RANKL in osteoclast-mediated resorption of bone makes this factor central to the process of physiologic bone remodeling that is under the primary control of 1,25(OH)₂D₃ and PTH. Unfortunately, the increased expression of RANKL by high or toxic levels of 1,25(OH)₂D₃ and PTH as well as by cytokines released locally in response to inflammation also makes this molecule generally responsible for a multitude of bone calcium mobilizing diseases several of which lead to osteoporosis. These diseases include those associated with vitamin D or PTH administration as well as menopause, andropause, and a variety of additional osteolytic states associated with arthritis, cancer and skeletal and dental implant wear debris. Indeed, the increased activity of RANKL together with additional bone active cytokines has now been implicated in the pathology of bone loss associated with an enormous list of diseases. These diseases include those of aberrant mechanical stress, systemic bone disease, Paget's disease, periodontal disease, skeletal neuropathy, systemic bone disease, postmenopausal osteoporosis, male osteoporosis, rheumatoid arthritis, osteoarthritis, diabetic neuropathy, multiple myeloma, follicular lymphoma, osteolytic disease due to metastatic cancer of the breast and other organs, general hypercalcemia of malignancy, treatment-induced bone resorption (retinoic acid- and doxirubicin-induced bone resorption), prosthetic joint loosening, and likely many others with which bone loss is associated.

Importantly, it is the induction of RANKL by 1,25(OH)₂D₃ that leads to the hypercalcemia that currently prevents the therapeutic use of this hormone and many of its active analogs for indications such as psoriasis, autoimmune disease and cancer. All efforts to understand how hormones such as vitamin D and PTH as well as other regulators such as the inflammatory cytokines modulate RANKL gene expression from osteoblasts (as well as other key target cells) have been thus far unsuccessful, largely because proximal promoter regions of the RANKL gene fail to respond in standard evaluation assays.

In recent unpublished work, however, the present inventors have used a new approach which has revealed regions within the RANKL gene that are responsible for regulation by 1,25(OH)₂D₃, glucocorticoids and likely other regulatory factors. These regions are located at surprising distances from the RANKL promoter, making them virtually impossible to detect by standard methods. The inventors' work opens the route to the identification of, for example, small molecule agents capable of inhibiting the transcriptional expression of RANKL as an alternative to the development of proteins and/or antibodies to block RANKL actions which is now the current focus.

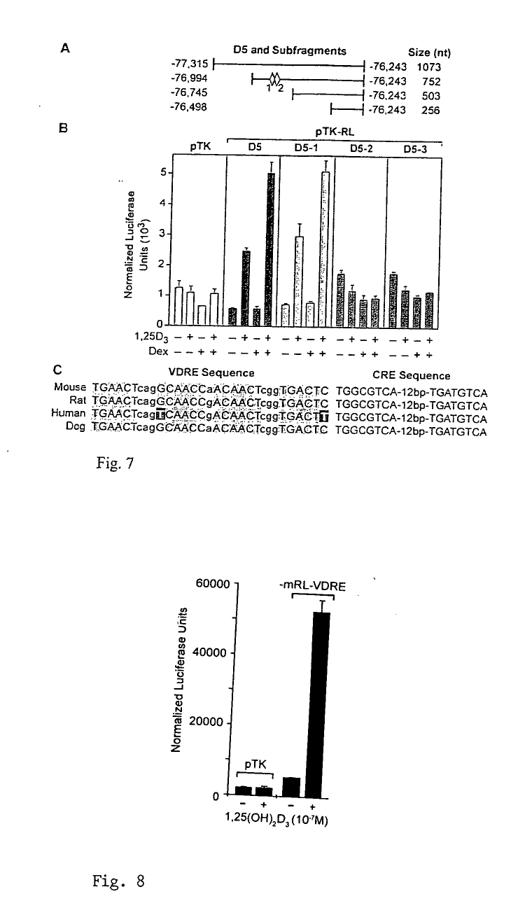

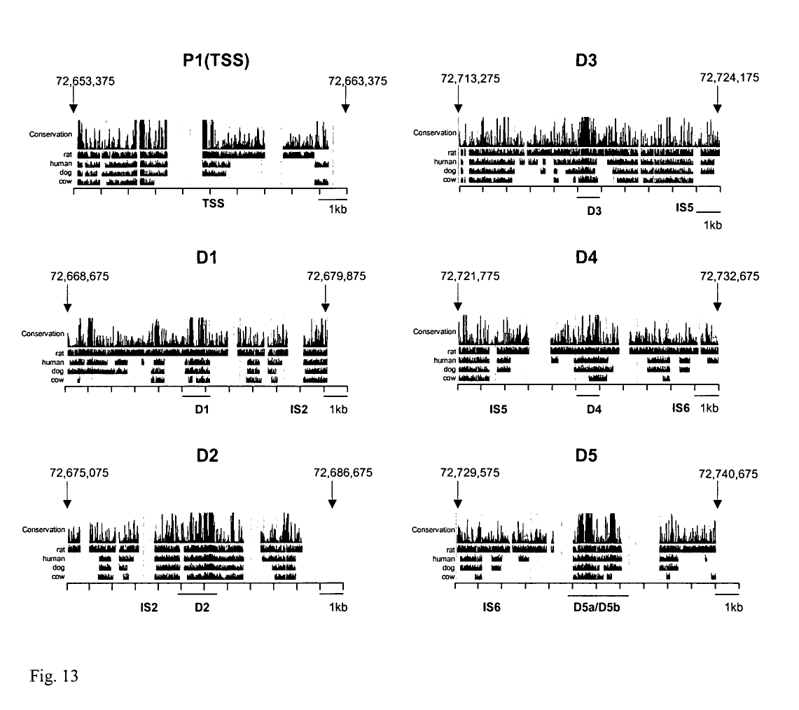

Accordingly, the inventors recently explored the RANKL gene promoter with the intent to identify regulatory regions using the technique of chromatin immunoprecipitation (ChIP) linked to DNA microarray analysis (chip). This procedure is termed ChIP/chip. Using this procedure, the inventors identified five regions of the mouse RANKL gene promoter which accumulate the vitamin D receptor when the cells are treated with 1,25(OH)₂D₃. These regions lie far upstream of the start site of transcription and bind in a time dependent fashion not only the vitamin D receptor and its partner RXR but the glucocorticoid receptor and the transcription factor C/EBPbeta as well. Each of these five regions was cloned, introduced into a reporter gene plasmid and evaluated for similar response to 1,25(OH)₂D₃ and glucocorticoids following transfection. The upstream region which bound the vitamin D receptor most avidly in intact cells displayed a striking response to the above hormones. Although the other 4 regions (D1-D4) did not show initial response to 1,25(OH)₂D₃, the addition of exogenous VDR lead to modest response from both D2 and D3. Subsequent mapping studies of this RL-enhanceosome have identified the 31 nucleotide sequence that represents two adjacent binding sites for the vitamin D receptor. FIG. 7 demonstrates the high transcriptional activity of this isolated VDRE in the context of the TK promoter. The response to glucocorticoids as well as OSM also maps to this mRL-D5 regions of the RANKL gene. OSM regulation indicates potential response to a cast of cytokines including the inflammatory cytokine IL-6. Importantly, each of these regions is conserved in the human RANKL gene, including the position and sequence of the VDRE the inventors have identified. FIG. 13 depicts sequence conservation among species across the D1 as well as the D2-D5 regulatory regions of the RANKL gene. Conservation in the TSS region as well as the D1-D5 regions of the RANKL gene in mouse, rat, human, and canine are indicated. Position and lack of conservation at intervening regions IS2, -5, and -6 are also shown in the figure.

The present inventors have discovered a region within the RANKL gene that mediates response to both calciotropic hormones as well as other factors such as the inflammatory cytokines and growth factors. Based on this work, high throughput screens are now possible to identify small molecules capable of regulating the expression of RANKL. It is clear that suppression of RANKL represents a viable mechanism whereby bone loss associated with a wide variety of natural inducers can be ameliorated and perhaps prevented.

Accordingly, the present invention provides an isolated nucleic acid including a polynucleotide sequence from the RANKL gene upstream region that is capable of conferring transcriptional responsiveness on a RANKL gene promoter. Table 1 sets forth particular RANKL upstream regions described and claimed herein. However, the numbering of particularly claimed sequences relative to transcriptional start sites may change due to currently un-sequenced components of the mouse and human RANKL genes within chromosome 14 (mouse) and chromosome 13 (human), respectively. Mouse sequence numbering (*) herein is based upon the transcriptional start site designated in the March 2005 Assembly, Build 34.1, Ensemble version 35 (November 2005). The mouse RANKL gene is transcribed on the reverse strand. Human sequence numbering (#) herein is based upon the transcriptional start site designated in the Ensemble version 35 (November 2005). The human RANKL gene is transcribed on the forward strand. The single DNA strand sequences provided in the Sequence Listing are, however, shown oriented 5′ to 3′ relative to the start site of transcription. Nucleotide numbers upstream of the transcriptional start site (TSS) are represented by negative (−) numbers. Based on the high degree of sequence conservation between the regions D1-D5, as illustrated in FIG. 13 and described herein, the RANKL upstream regions of the invention encompass corresponding RANKL upstream regions having substantial sequence homology and shall not be limited to the specific examples set forth herein; the present invention encompasses additional RANKL gene upstream sequences which have de minimus sequence variations from, and retain substantially the same functions relative to RANKL gene regulation, as the actual sequences disclosed and claimed herein.

TABLE 1

| Upstream RANKL | | |
|---|---|---|
| region | Mouse(Chrom 14)* | Human(Chrom 13)^{#} |
| D5 | −78,100 to −76,200 | −99,247 to −95,753 |
| | (SEQ ID NO: 5) | (SEQ ID NO: 10) |
| D4 | −69,300 to −68,400 | −87,514 to −86,532 |
| | (SEQ ID NO: 4) | (SEQ ID NO: 9) |
| D3 | −60,800 to −59,900 | −75,273 to −74,549 |
| | (SEQ ID NO: 3) | (SEQ ID NO: 8) |
| D2 | −23,300 to −21,700 | −25,569 to −24,246 |
| | (SEQ ID NO: 2) | (SEQ ID NO: 7) |
| D1 | −16,500 to −15,300 | −21,079 to −20,018 |
| | (SEQ ID NO: 1) | (SEQ ID NO: 6) |

In certain nucleic acids, a promoter is included in the molecule that is subject to transcriptional activation and/or repression by the RANKL upstream polynucleotide sequence. The promoter may be the native RANKL gene promoter, in an isolated minimal form, or, alternatively, a promoter not naturally under control of RANKL gene enhancer elements. Suitable non-native promoters include, for example, the Herpes simplex virus thymidine kinase promoter.

With respect to the promoter which is part of a transcriptional control region of the invention, practically any promoter may be used, so long as the transcriptional activity of such a promoter can be modulated by a response element of the present invention (when suitably provided or positioned in operative fashion relative to the promoter). Presently preferred are promoters which require a response element for activity, including, but not limited to, the response elements for C/EBPbeta. VDR, GR, Stat3, or CREB. As those of ordinary skill in the art will understand, the response elements of the present invention, like other response elements, are orientation and, with wide latitude, position independent. Thus, the response elements of the present invention are functional in either orientation and may be placed in any convenient location from the promoter to be affected.

In more preferred embodiments, a reporter gene operatively linked for transcription by the promoter is further included in the nucleic acid. Suitable reporter genes include, but are not limited to, reporter gene encoding luciferase, chloramphenicol acetyl transferase, beta-lactamase, green fluorescent protein, or beta-galactosidase, with luciferase being the most preferred reporter gene.

In another embodiment, the invention provides a host cell comprising an isolated nucleic acid as described and claimed herein, preferably a host cell endogenously-expressing a transcriptional factor or steroid/thyroid hormone receptor) capable of interacting with the RANKL upstream region to confer transcriptional responsiveness on an operatively linked promoter. Certain preferred host cells comprise a RANKL upstream region according to the invention and a reporter gene operably linked to a promoter that is not normally subject to transcriptional activation and/or repression by the RANKL upstream region.

Preferred cells for use with expression systems employing transcriptional control regions under control of response elements according to the invention are mammalian cells, including, but not limited to, mouse ST2 or human osteoblastic cell lines such as MG-63 or any cell line that expresses RANKL such as certain fibroblasts, synoviocytes, and T cells. Host cells according to the invention are preferably capable of expressing endogenous transcription factors or members of the steroid/thyroid superfamily of hormone receptors, most preferably vitamin D₃ receptor. Thus, via gene transfer with appropriate expression vectors comprising a heterologous gene under the control of a transcriptional control region of the invention, it is possible to convert certain host cells into transformed cells which produce increased quantities of a desired protein in response to induction by, for example, a ligand for a member of the steroid/thyroid superfamily of receptors. It should also be noted that, alternatively, host cells need not endogenously express receptors but may be transfected with appropriate vectors which provide for the expression of a receptor or other transcriptional regulators of the artisan's choice.

Expression plasmids containing the SV40 origin of replication can propagate to high copy number in any host cell which expresses SV40 Tag. Thus, expression plasmids carrying the SV40 origin of replication can replicate in COS cells, but not in CV-1 cells. Although increased expression afforded by high copy number is desirable, it is not critical to the assay systems described herein. The use of any particular cell line as a host is also not critical, although mouse ST2 and human MG-63 cells are presently preferred because they are particularly convenient, as described in the Examples section.

In yet another embodiment, the invention provides a method for identifying a chemical entity capable of altering RANKL gene transcriptional activity. Such a method includes steps of: (a) providing a host cell containing a reporter gene operatively linked to a promoter that is transcriptionally responsive to a RANKL upstream region also present in the host cell; (b) exposing the host cell to a chemical entity; and (c) measuring and comparing reporter gene expression to that of a control cell that is not exposed to the chemical entity wherein a higher or lower expression level than that of a control cell indicates that the chemical entity is capable of altering RANKL gene transcriptional activity.

Test compounds contemplated for screening in accordance with the invention assay methods include any chemical entity which can potentially affect the ability of receptor to modulate transcription activity through a response element of the present invention. Candidate/test compounds include, for example, 1) peptides such as soluble peptides, including Ig-tailed fusion peptides and members of random peptide libraries (see, e.g., Lam, K. S. et a!. (1991) Nature 354:82-84; Houghten, R. et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang, Z. et al. (1993) Cell 72:767-778); 3) antibodies (e.g., polyclonal, monoclonal, humanized, anti-idiotypic, chimeric, and single chain antibodies as well as Fab, F(ab′)₂ Fab expression library fragments, and epitope-binding fragments of antibodies); and 4) small organic and inorganic molecules (e.g., molecules obtained from combinatorial and natural product libraries). Small molecules are particularly attractive candidate/test compounds because such chemical entities typically provide ease of delivery (e.g., oral administration) and their potency, efficacy and selectivity can be enhanced or modulated via deliberate chemical modification. Accordingly, methods directed at the identification of small molecules capable of altering RANKL gene transcriptional activity represent preferred embodiments of the invention.

Test compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam, K. S. (1997) Anticancer Drug Des. 12:145).

Examples of methods for the synthesis of molecular libraries can be found in the art, for example, in: DeWitt et al. (1993) Proc. Natl. Acad. Sci. U.S.A. 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed, Engl. 33:2061; and Gallop et al. (1994) J. Med. Chem. 37:1233.

The invention is also directed to a method for identifying a chemical entity having reduced hypercalcemic activity. Such a method includes steps of: (a) providing a host cell containing a reporter gene operatively linked to a promoter that is transcriptionally responsive to a RANKL upstream region also present in the host cell; (b) exposing the cell to a chemical entity; and (c) measuring and comparing reporter gene expression to that of a control cell treated with a known hypercalcemic agent wherein a lower expression level than that of the control cell indicates that the chemical entity possesses reduced hypercalcemic activity. In a preferred embodiment, the known hypercalcemic agent is 1,25(OH)₂D₃ and the chemical entity is a vitamin D analog.

Yet another embodiment of the invention is directed to a method for the controlled expression of a gene. Such a method includes the steps of: (a) providing a host cell containing a RANKL upstream region further containing a gene operatively linked for transcription to a promoter that is transcriptionally responsive to a RANKL upstream region also contained within the host cell ; and (b) culturing the host cell under conditions to express the gene. In certain embodiments, the host cell endogenously expresses a steroid/thyroid hormone receptor or other transcription factor capable of interacting with the RANKL upstream region and regulating RANKL gene expression (e.g., the vitamin D₃ receptor).

The following examples are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### III. EXAMPLES

### Example 1

### Establishing the RANKL Expression Model in Mouse ST2 Cells

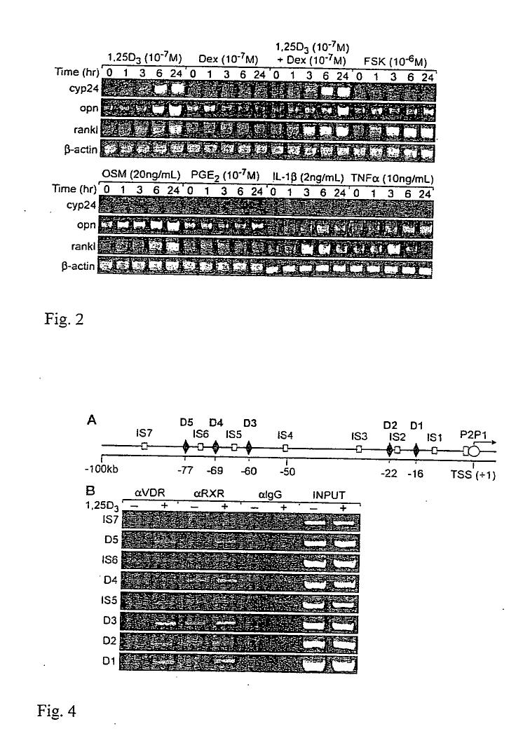

The inventors first established a model in which RANKL is both induced in response to 1,25(OH)₂D₃, PTH (forskolin) and oncostatin M (OSM), and is capable of supporting osteoclast formation when co-cultured with bone marrow monocyte- or spleen cell-derived osteoclast precursors. While a number of cell types express RANKL and induce osteoclastogenesis, the historical literature strongly supports mouse ST2 cells in this capacity, prompting the inventors to focus on this stromal/preosteoblastic line for initial studies. As seen in FIG. 2, treatment of ST2 cells with 1,25(OH)₂D₃, forskolin (PTH mimic), OSM, and PGE2, as well as both IL-1beta and TNFalpha leads to a substantial time-dependent increase in RANKL transcripts as compared to β-actin controls. Each of these components is known to both induce RANKL expression and promote osteoclast formation as well. 1,25(OH)₂D₃ also induced in a time-dependent manner both Cyp24 and osteopontin (OPN), gene targets the inventors used as controls. The inventors also examined the effects of glucocorticoids (GC) such as dexamethasone (Dex), a steroid that together with 1,25(OH)₂D₃ is required for induction of osteoclast formation by ST2 cells. Indeed, GC effects are widely observed. As can be seen, while having little or no effect on RANKL expression alone, Dex enhanced the activity of 1,25(OH)₂D₃ on RANKL expression. The ability of GCs to enhance osteoclast formation in coculture assays in the presence of 1,25(OH)₂D₃, however, is likely due also to its coordinate ability to suppress OPG. These data confirm the ST2 cells as a viable model to explore the molecular regulation of RANKL by key hormones and cytokines in vitro.

### Example 2

### Identifying Regions Within the Mouse RANKL Gene with Potential Vitamin D Regulatory Capability

The lack of convincing data with regard to RANKL regulation by 1,25(OH)₂D₃ and its receptor (VDR) as well as the absence of data for regulation by other transcription factors led the inventors to explore the RANKL gene for transcription factors binding sites using the approach described above. This approach, termed ChIP/chip, involves isolating and enriching RANKL promoter DNA using ChIP and then using this DNA to screen a DNA microarray containing tiled 50-mer oligonucleotides that span in a contiguous manner a large region surrounding the RANKL gene locus. The inventors focused initially upon localizing the VDR/RXR heterodimer largely because of the abundance of recognized osteoblastic target genes for 1,25(OH)₂D₃ useable as controls.

a. ChIP.

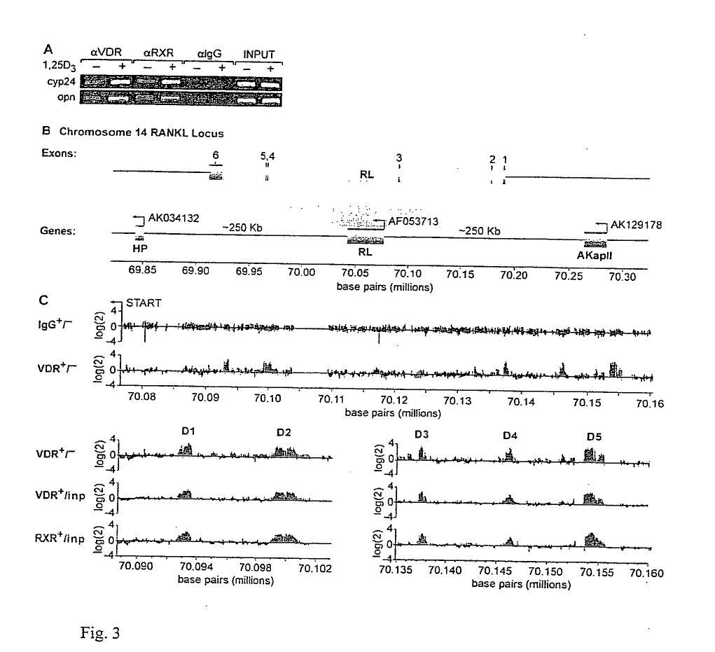

As a first step, the inventors treated ST2 cells with either vehicle or 1,25(OH)₂D₃ for 6 hours, fixed the cells with formalin, and then subjected the cell lysates to ChIP analysis using antibodies to VDR, RXR, and an IgG control. To confirm that the ChIP assay produced enriched DNA for 1,25(OH)₂D₃ target gene promoters, the inventors amplified the DNA for the VDRE-containing promoter regions of both Cyp24 and OPN. As observed in FIG. 3a, precipitation using antibodies to the VDR and RXR, but not IgG, clearly increased the abundance of DNA for both Cyp24 and OPN following treatment with 1,25(OH)₂D₃. Immunoprecipitated DNA from these six conditions plus input DNA (eight samples) were then amplified using linear, ligation-mediated PCR and utilized in the DNA chip studies to identify VDR/RXR binding sites within the RANKL gene.

b. DNA Microarrays (Chip).

The RANKL gene locus located on mouse chromosome 14 is seen in FIG. 3b, and is transcribed on the reverse strand beginning at nucleotide 70,077,208 and ends at nucleotide 70,042,000. Genes flanking RANKL include AU021034 (an expressed sequence tag), located some 250 kilobases downstream and AKap11 (AK129178), located some 250 kilobases upstream of the RANKL locus. Thus, RANKL is bounded on each side by over 250 kilobases of intergenic DNA. Custom Nimblegen oligonucleotide microarrays were therefore prepared consisting of a tiling of 50-mer oligonucleotides that spanned the mouse RANKL gene beginning over 200 kilobases upstream of the TSS to over 100 kilobases downstream of the final exon. The tiling span was synthesized in duplicate in both the forward (5′ to 3′) as well as in the reverse (3′ to 5″) direction, thus providing 4 independent readings for each oligonucleotide site. Tiled oligonucleotides spanning positive control gene loci and a number of other potential target gene loci were also synthesized due to the enormous capacity of the DNA chip (some 380,000 usable sites). The following comparisons were made using combined Cy3 and Cy5 labeled DNA's: VDR(1,25(OH)₂D₃ treated) vs VDR(vehicle), VDR(1,25(OH)₂D₃ treated) vs Input DNA, RXR(1,25(OH)₂D₃ treated) vs RXR (vehicle), RXR (1,25(OH)₂D₃ treated) vs Input DNA, IgG(1,25(OH)₂D₃ treated) vs IgG(vehicle), and IgG(1,25(OH)₂D₃ treated) vs Input DNA. A summary of the data is seen in FIG. 3c, which due to space limitations represents only a focused view of the fluorescence data (log2) generated for the portion of the RANKL gene extending upstream of the TSS. As can be seen, both VDR and RXR bind to five regions of the RANKL gene located some −16, −22, −60, −69 and −77 kilobases upstream of the TSS. These regions were designated D1 (−16), D2 (−22), D3 (−60), D4 (−69) and D5 (−77). Interestingly, no significant binding was observed at the previously identified proximal promoter region located at −1 kilobase from the RANKL TSS. No fluorescent peaks were similarly observed for VDR or RXR either further upstream of −77 or downstream of the TSS. Binding was also not observed when a comparison of IgG to input DNA was performed. Importantly, however, VDR and RXR localization was identified at the expected sites in control gene loci, specifically those associated with both Cyp24 and OPN. These results suggested to us that 1,25(OH)₂D₃ might regulate the RANKL gene at multiple sites located unusual “distances” from the RANKL TSS and detectable only by the methods that the inventors employed.

c. DNA Microarray Confirmation.

To confirm binding of the VDR and RXR to the RANKL gene selectively at these five sites, the inventors treated ST2 cells in repeated experiments with either vehicle or 1,25(OH)₂D₃ for 6 hours, fixed the cells, and then subjected them again to direct ChIP analysis using antibodies to VDR, RXR and IgG. The isolated DNA was then evaluated by PCR using primers as depicted in the diagram seen in FIG. 4a which were designed to amplify small DNA segments within the 5 regions as well as segments located at intervening sites (IS) outside the five regions. As observed in FIG. 4b, although VDR and RXR were not found associated with IS5, IS6 or IS7 of the RANKL gene at −66, −72, and −88 kilobases, their binding was clearly enriched in response to 1,25(OH)₂D₃ at sites within the five regions identified in the ChIP/chip analysis at −16 (D1), −22 (D2), −60 (D3), −69 (D4), and −77 (D5) kilobases from the RANKL TSS. No binding was also observed at additional intervening regions located at −55, −31, −21, and −10 kilobases. Additional analyses confirmed the binding of VDR at −69 (D4), since VDR binding was not as evident at the −69 kb region in the experiment in FIG. 4b as seen at the other 4 sites. Interestingly, the binding of the VDR at D4 was also the weakest of all the sites identified in the microarray scanning experiment as well (FIG. 3c). These data confirm the presence of the VDR/RXR heterodimer at five independent sites (D1-D5) on the RANKL gene. It is clear from these as well as many additional ChIP experiments the inventors have performed, however, that the most striking interaction between the VDR/RXR heterodimer and the RANKL gene occurred at D5. For reasons that will become apparent later, the inventors have designated the D5 region and vicinity the RANKL central control locus or RL-CCL.

### Example 3

### Glucocorticoids Promote Glucocorticoid Receptor (GR) Binding and Enhance Both C/EBPbeta and VDR/RXR Binding to the RANKL Gene

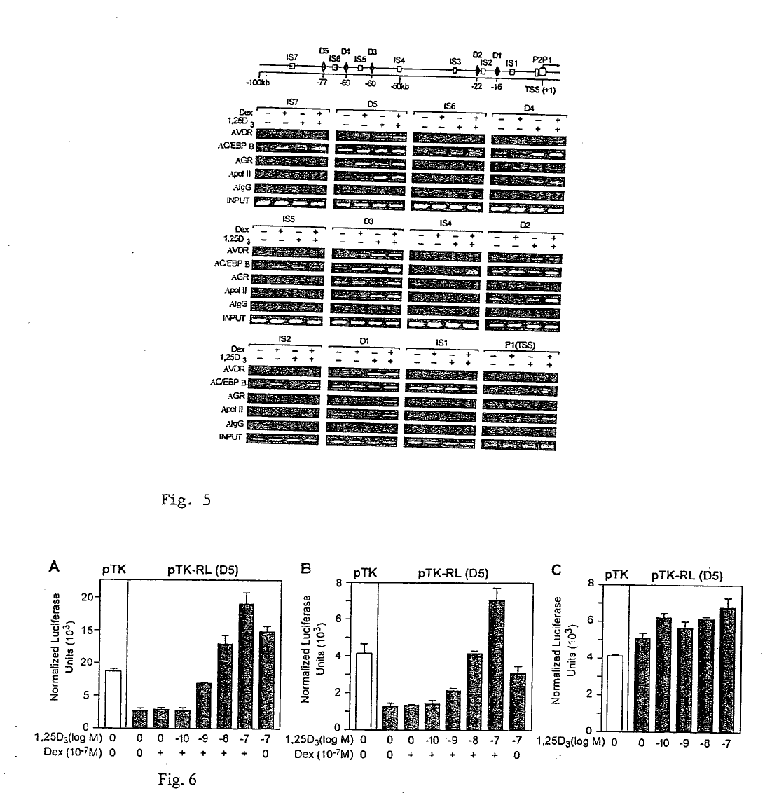

The inventors' studies at the level of RANKL mRNA suggest that GCs can facilitate the ability of 1,25(OH)₂D₃ to induce RANKL expression, although the mechanism remains unknown. The inventors hypothesized that GC's might perhaps enhance VDR binding to RANKL regulatory regions. To address this, they treated ST2 cells with either vehicle, 1,25(OH)₂D₃, Dex or both 1,25(OH)₂D₃ and Dex, and after 6 hours subjected the groups to ChIP analysis using antibodies to VDR, GR, C/EBPbeta or IgG. The inventors focused on the RL-CCL region (D5) and amplified the precipitated DNA using primers to this region of the gene as well as to D4, D3 and TSS and intervening regions. As can be seen in FIG. 5, although Dex had little or no effect on VDR binding in the absence of 1,25(OH)₂D₃, it modestly increased the binding of the VDR to D5, D4, and D3 when used in combination with the vitamin D hormone. Perhaps not surprisingly, Dex also stimulated the binding of GR to these regions, supporting the idea that GR may directly mediate the activity of GCs through potential GR binding sites located within these regulatory regions of the RANKL gene. The inventors also explored the possibility that C/EBPbeta might similarly participate in the RANKL activation process. C/EBPbeta plays a significant role in the regulation of osteoblastic genes, and is found associated with both 1,25(OH)₂D₃ and GC target genes. As can be seen in FIG. 5, C/EBPbeta was indeed localized to the RL-CCL, D4 and D3. It was present, however, both in the absence as well as in the presence of the inducers, and modestly upregulated when Dex was present. Finally, the inventors observed the presence of RNA pol II (FIG. 5). Surprisingly, this enzyme was recruited into not only the TSS regions but to the RL-CCL region and the D4 and D3 regions as well. The binding of VDR, GR and C/EBPbeta to the RANKL gene is highly reminiscent of many other genes that are activated by GCs, suggesting that a potential transcription factor regulosome may be operable on the RANKL gene. These data strongly support the idea that the D5 region, while located far upstream of the RANKL TSS, does indeed function to regulate RANKL gene expression. Although possible, it seems unlikely that the ability of 1,25(OH)₂D₃ and the GCs to induce both VDR/RXR and GR binding to the RL-CCL and to induce RANKL mRNA would be unrelated.

### Example 4

### Examining the Transcriptional Activity of D5 in the Context of the Viral Thymidine Kinase Promoter

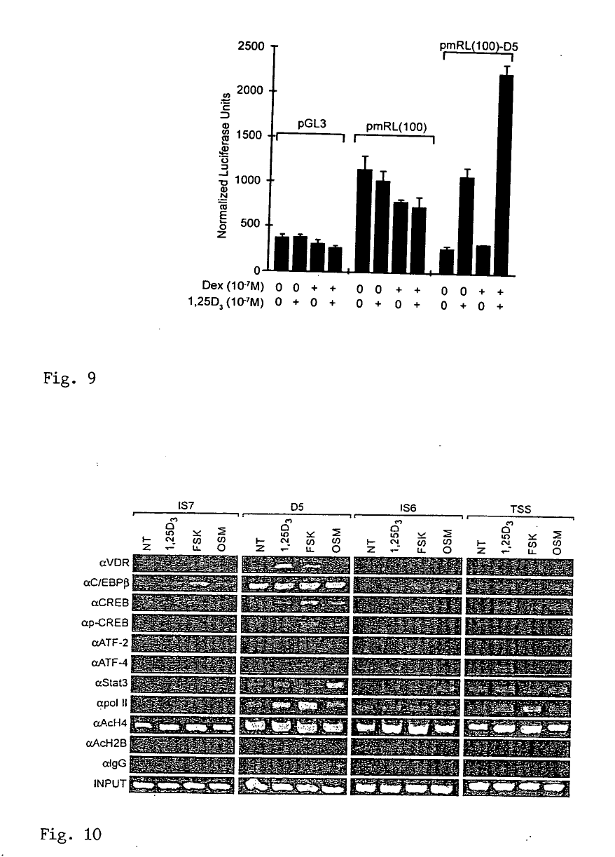

The activity of an enhancer must be evaluated in the context of its local environment, preferentially in the context of both other enhancers and its native promoter. In the case of the RANKL gene, however, this is clearly not possible due to the numbers and the distant locations of the potential regulatory regions. To tackle this problem, the inventors isolated approximately 800 to 1100 bp fragments of each region (D1 to D5) as identified through the ChIP/Chip analysis, cloned each into a luciferase expression vector under the control of a viral thymidine kinase (TK) minimal promoter, and explored the 1,25(OH)₂D₃- and GC-inducible activity of each following transfection into ST2 cells. This approach has some limitations, particularly if fragments fail to exhibit inducible activity. Nevertheless, positive results provide added support for the potential regulatory capabilities of an individual DNA fragment even when examined in isolation. Regions D1 through D4 failed to show increased activity when the ST2 cells were treated with increasing concentrations of 1,25(OH)₂D₃. As observed in FIG. 6a, however, D5 (RL-CCL) manifested a typical and rather striking dose response to 1,25(OH)₂D₃ when introduced into ST2 cells both in the absence as well as in the presence of Dex. Importantly, while Dex had only a marginal effect on its own, the activity of 1,25(OH)₂D₃ was potentiated in its presence, an effect that was previously observed both at the level of RL mRNA induction (FIG. 2) and at the level of VDR binding to the RL-CCL region (FIG. 5). The pTK-luc vector showed no response to the presence of the hormones (see also FIG. 7). This induction was magnified by the addition of a VDR expression vector (FIG. 6a), but was also evident in the absence of such addition (FIG. 6b). Added VDR expression vector was also capable of inducing a modest response to D2 and D3 but not D1 or D4. Interestingly, 1,25(OH)₂D₃ failed to stimulate via the D5 fragment (pRL-D5/TK-luc) when it was introduced into COS7 cells together with VDR expression vector (FIG. 6c) as well as in the osteoblast mouse cell line MC-3T3-E1 which does not produce RANKL. These data suggest that this region and perhaps additional sequences adjacent to D5 contain a binding site responsible for tissue-specific as well as hormone-inducible expression of RANKL. For this reason, the inventors have termed this region the RANKL gene the central control locus or RL-CCL. In fact, inspection of the initial ChIP/chip data (FIG. 3c) together with numerous follow-up ChIP experiments suggest that VDR binding in these regions is indeed significantly weaker. In that context, it is possible that these regions provide regulatory loci that are only capable of functioning in combination and/or in context with the RANKL promoter. It seems clear that the D5 region together with the upstream mRL-D5b regions , termed the RL-CCL, regains significant regulatory capability in the context of the TK promoter. Importantly, the mRL-D5 and mRLD5b regions also manifest similar response when cloned into the native endogenous mouse RANKL promoter with coordinates −100 to +56 nucleotides. To ensure that the viral TK promoter background was not contributing to our evaluation, the inventors cloned the mouse RANKL promoter (−100 to +65) into pGL3, inserted RL D5 and evaluated its activity in ST2 cells as well. The results documented in FIG. 9 indicate that while the minimal RANKL promoter is unresponsive to 1,25(OH)₂D₃ or Dex, insertion of D5 leads to a transcriptional response identical to that seen in the TK background.

Transcriptional activities of the D1-D4 regions of the RANKL gene in the context of the TK promoter were analyzed and corresponding data is shown in FIG. 12. The D1-D4 regions of the RANKL gene were cloned into the pTK vector and termed pTK-RL(D1)-(D4). ST2 cells were transfected with pTK-luc or the pTK-RL(D1-D4) constructs and a VDR expression vector and then treated with either vehicle, 1,25(OH)₂D₃, Dex or both. Cells were harvested after 24 hours and luciferase activity assessed. Luciferase was normalized using beta-gal activity. Data in FIG. 12 represent the mean of triplicate determinations (SEM).

### Example 5

### Mapping Response to 1,25(OH)₂D₃ in the RL-CCL

ChIP data are of inherent low resolution, making it difficult to identify the specific sequence to which the VDR/RXR heterodimer binds within the RL-CCL. 1,25(OH)₂D₃-inducibility of the RL-CCL DNA fragment, however, provides the opportunity to precisely map the location of the VDRE using mutagenesis. The inventors therefore subjected the RL-CCL DNA fragment to 5′ deletion analysis using PCR and prepared a series of fragments of the RL-CCL with increasingly fore-shortened 5′ termini as illustrated in FIG. 7a. These RANKL gene fragments were similarly cloned into the pTK-luc vector and their activities together with the wildtype D5 fragment examined in response to 1,25(OH)₂D₃, glucocorticoids and the combination following transfection into ST2 cells. The results in FIG. 7b show that while the typical Dex-enhanced 1,25(OH)₂D₃ response observed previously (FIG. 5) was retained in both the wildtype 1072 bp D5 fragment and the 752 bp D5-1 fragment, the 1,25(OH)₂D₃ response was loss in the subsequent D5 fragments D5-2 and D5-3. These results indicate that a potential RANKL VDRE is likely to be present between nucleotides −76994 and −76745 relative to the RANKL TSS. Indeed, inspection of this region without the aid of an in silico program revealed a potential complex VDRE sequence located at −76,892 upstream of the RANKL TSS. Cloning of this 31 bp VDRE sequence into the TK promoter revealed strong response to 1,25(OH)₂D₃, as shown in FIG. 8. Interestingly, this potential VDRE sequence is fully conserved across all species tested as documented in FIG. 7c. As well, this region of the RL-CCL also contains a number of consensus GR binding half-sites, and at least one consensus GRE duplex site.

### Example 6

### The RL-CCL Region in Regard to CREB and Stat3 Binding in Response to Forskolin and OSM

In addition to 1,25(OH)₂D₃ and GCs, RANKL expression is also known to be modulated by such factors as PTH, PGE2, the gp130-activating cytokines IL-6 and OSM, as well as IL-1beta and TNFalpha. Indeed, the results of the experiment depicted in FIG. 2 confirmed upregulation of RANKL in response to each of these agents in ST2 cells. Forskolin was used as substitute for PTH, however, as ST2 cells are deficient in PTH receptor (PTHR). Both PTH/forskolin and PGE2 are known to induce RANKL gene expression via CREB whereas OSM functions via Stat3. Moreover, both forskolin and OSM are capable of promoting osteoclast formation in co-culture assays, indicating that each is sufficient in and of itself. The inventors therefore explored the possibility that the RL-CCL might represent a focal target for these inducers via either CREB or Stat3 in the RANKL gene locus. ST2 cells were treated with either vehicle, 1,25(OH)₂D₃, forskolin or OSM for a 6 hour period and then subjected to a ChIP analysis using antibodies to VDR, C/EBPbeta, CREB, phospho-CREB, ATF-2, ATF-4, Stat3, RNA pol II and control IgG. Isolated DNA was then subjected to amplification using primers corresponding to the D5 region (RL-CCL) of the RANKL gene. As controls, the inventors also assessed the extent to which the immunoprecipitations led to the enrichment of DNA at sites surrounding the D5 region of the RANKL gene. As can be seen in FIG. 10, 1,25(OH)₂D₃ induced the binding of the VDR to the D5 region of the RANKL gene as expected. Treatment with forskolin also induced a small amount of VDR binding in the absence of 1,25(OH)₂D₃. The inventors have observed this increase in basal VDR binding in response to forskolin on the osteopontin gene (but not the Cyp24 gene) in the absence of ligand as well. OSM had no effect on VDR binding to the D5 region, as expected. Forskolin, in contrast, stimulated the binding of CREB to the D5 region of the RANKL gene, but did not activate either ATF-2 or ATF-4, related members of the CREB family of transcription factors. CREB binding in response to forskolin was also associated with an enhanced level of its phosphorylation (PCREB) (FIG. 10). Additional studies using ChIP analysis show that CREB and phosphoCREb bind preferentially upstream of D5 in the D5b region whereas Stat3 binds preferentially to the D5 regions alone. Unexpectedly, both 1,25(OH)₂D₃ and OSM also modestly increased CREB association at the D5 site. This potential involvement of CREB in the activation of RANKL by 1,25(OH)₂D₃ and OSM activation may provide the molecular basis for the observation made by O'Brien and coworkers that a dominant negative CREB protein inhibits not only PTH-induced RANKL expression but suppressed the ability of 1,25(OH)₂D₃ and OSM to induce RANKL mRNA as well. Stimulation of ST2 cells with OSM induces significant binding of Stat3 to the D5 region of the RANKL gene. Stat3 binding to the D5 region is not observed in response to forskolin, although some Stat3 binding can be seen in response to 1,25(OH)₂D₃. Since these results are highly reproducible, they suggest selective cross-talk among the signaling pathways that activate transcription factors such as CREB, Stat3 and perhaps VDR. These data support the possibility that the D5 and D5b region is a central control locus (CCL) responsible for activation by a number of unrelated regulatory factors.

### Example 7

### Elements in the Cloned D5 Region Mediate Both 1,25(OH)₂D₃ and OSM but not Forskolin Activity in the RANKL Gene Locus

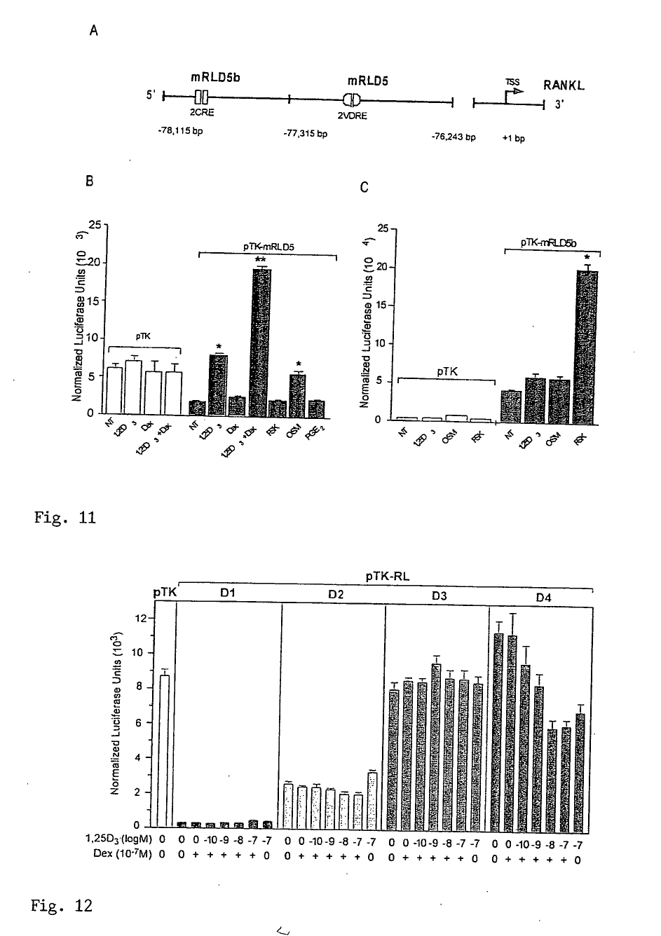

Based upon the above data, the inventors explored the possibility that similar to 1,25(OH)₂D₃, forskolin, PGE2 and OSM might be capable of activating transcription via the D5 fragment of the RANKL gene. The respective fragments and their designation are seen in FIG. 11a. The inventors introduced the mRL-D5 TK-luc plasmid or the mRL-D5b TK-luc plasmid into ST2 cells via transfection and then stimulated the cells with either vehicle, 1,25(OH)₂D₃, Dex, 1,25(OH)₂D₃ and Dex, forskolin, PGE2 and OSM. Cells were harvested 24 hours later and evaluated for luciferase activity as in the previous experiments. As can be seen in FIG. 11b, OSM but not forskolin or PGE2 was able to induce a reproducible 3 fold increase in transcriptional output via mRL-D5. The effects of 1,25(OH)₂D₃ and the potentiating effects of Dex were again observed. As seen in FIG. 11c, the mRL-D5b region, however, was responsive to forskolin but not 1,25(OH)₂D₃ or OSM. The presence of two CREB response elements (CREs) identified in silico are present in this regions. Examination of the region immediately upstream of mRL-D5 (termed mRL-D5b and part of the regions designated RL-CCL) demonstrates direct response to forskolin. The presence of two CREB response elements (CREs) identified in silico are present in this region. As seen in FIG. 15, point mutation of each of these sites leads to loss of forskolin response, indicating that these CREs mediate forskolin (and likely PTH) response.

### Example 8

### Preparation of Osteoblastic Cell Lines Containing Genetically Stable Reporter Genes Under the Control of Human RANKL Transcriptional Regulatory Regions

The human FOB cell line may be used to prepare stable clones containing integrated copies of the human RANKL regulatory region(s) fused to the reporter gene luciferase. Regions of the human RANKL gene demonstrated to be key to hormonal and cytokine regulation are introduced into the hFOB cell line together with a neomycin-selectable gene marker and viable clones selected under conditions of 400 mg/ml of the drug G418. At least 10 cell clones that exhibit satisfactory growth conditions in normal culture medium containing 10% fetal bovine serum and 400 mg/ml G418 are evaluated further for each construct. As an initial characterizing screen, the clonal cell lines are treated with 1,25(OH)₂D₃ and the ability of this hormone to induce luciferase activity assesses after 16 to 24 hours. Both the ability to respond to 1,25(OH)₂D₃ and the extent/magnitude of response (fold induction) are used to determine which of the clonal lines exhibit traits most favorable to further analyses. Alternatively, these human RANKL regulatory regions may be introduced into the human osteoblastic MG-63 cell line or the mouse ST2 cell line and cell clones selected under identical conditions. It seems likely that the host species cell background in this case is not likely to be a significant determining factor. The lines are established for continuity and large numbers of cells cryopreserved for future use.

### Example 9

### Characterizing the RANKL Regulatory Region Stable Cell Lines for Hormonal/Cytokine Response

Having established clonal cell lines that contain relevant human RANKL regulatory regions fused to luciferase stably integrated into the hFOB genome, these lines are further characterized for responsiveness to not only 1,25(OH)₂D₃, but to PTH, IL-1, oncostatin M and PGE2 as described in this example section and known in the art. Cells may be treated with the various hormones and/or cytokines or prostaglandin at maximum concentrations and the activities of these hormones via the RANKL regulatory region(s) assessed after 24 hours.

### Example 10

### Vitamin D and Glucocorticoids Modulate the Expression of the RANKL Gene in Human Cells

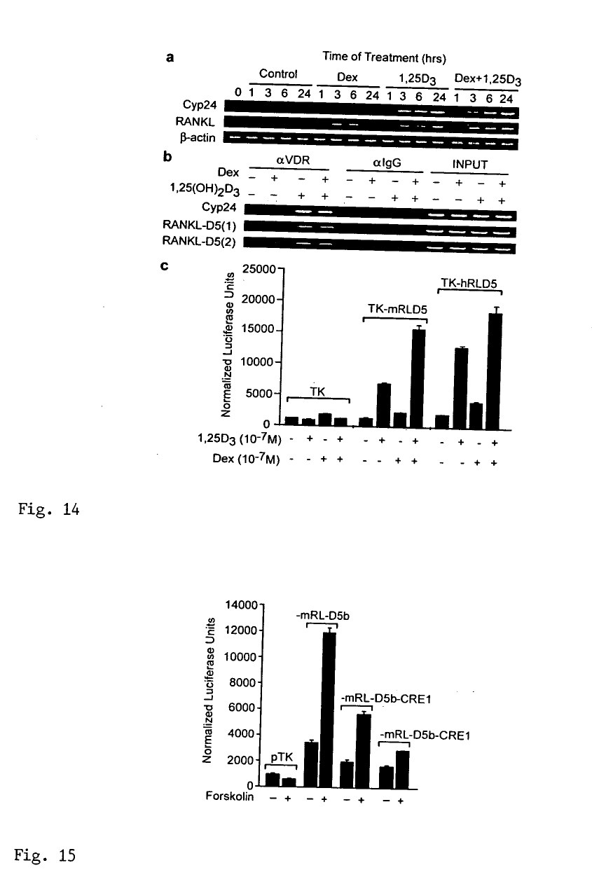

The human osteoblastic cell line MG-63 was treated with various combinations of 1,25(OH)₂D₃ and/or glucocorticoids (dexamethasone, dex)) for increasing periods of time. The isolated RNA was then subjected to RT-PCR analysis using primers to a control gene (β-actin), a known vitamin D induced gene (Cyp24) and RANKL. As can be seen in FIG. 14a, RANKL is upregulated in response to both 1,25(OH)₂D₃, dex and the combination. These and additional data using other hormonal regulators are identical to that observed in mouse cells and it therefore appears likely that human RANKL is regulated in a fashion similar to that in mouse.

MG-63 cells were treated with vehicle, 1,25(OH)₂D₃, dex, or the combination for 3 hours, and then subjected to ChIP analysis using primers to a control gene (Cyp24) or to the D5 region of the human RANKL gene (two different sets of primers). Input represents DNA abundance prior to precipitation with either antibodies to the VDR or non specific IgG. As can be seen in FIG. 14b, VDR binding to the D5 region of the human RANKL gene is induced in response to 1,25(OH)₂D₃, indicating that this conserved region in the human does indeed mediate the activities of vitamin D, the glucocorticoids and other regulators. The VDR heterodimer partner RXR is also induced to bind.

Approximately 1000 bp of the human RANKL gene comprising D5 was cloned and inserted into a TK-luciferase expression vector (TK-hRLD5). The vector was introduced in parallel with the mouse TK-mRLD5 region or the control vector (TK) into human MG-63 osteoblastic cells by transfection and its transcriptional activity after 24 hours determined in response to vehicle, 1,25(OH)₂D₃, Dex and the combination. As can be seen in FIG. 14c, 1,25(OH)₂D₃ induces and dex synergistically enhances the activity of both the mouse and human D5 regions in a similar fashion. This indicates that the regulatory sequences within these two D5 regions perform similar functions. The region identified in the human RANKL gene appears to behave identically to that which the inventors extensively characterized in the mouse gene and can be used in an analogous fashion to establish a drug screen. The D5 RL region and an additional region immediately upstream in both the mouse and human genes contain elements that mediate the actions of parathyroid hormone, a variety of prostaglandins, cytokines such as oncostatin M and IL-6, as well as IL-1 and others.

### Example 11

### Miniaturizing the RANKL Screening Assay for High Throughput Mode

This example describes establishing a high throughput screen for small molecules that might inhibit RANKL expression as well as to screen active vitamin D analogs for their transcriptional activity on the RANKL gene. Thus, the assay might represent an in vitro mechanism for assessing the bone calcemic potential for vitamin D analogs that is currently available only through in vivo studies. It is preferred to develop assays in the 96-well format that will provide sufficient luciferase output to be useful in an automated screen. An operable assay consists of first plating stable osteoblastic cells at optimal concentrations into individual wells of a 96-well plate and carrying out dose response curves with 1,25(OH)₂D₃ or other inducer and then determining the levels of both basal as well as inducible luciferase activity that are measurable using a standard luminometer.

The preparation and identification of cell lines potentially useful in the development of a high throughput screen for general agonists and more appropriately antagonists of RANKL expression will allow the practice of assays to characterize synthetic vitamin D ligands for their ability to activate or repress RANKL gene expression. Those incapable of such actions might represent a subset of vitamin D ligands that do not retain the capacity to promote bone calcium mobilization, a currently debilitating activity on the part of many highly potent vitamin D analogs.

Responsive cell lines should be treated with the activator/inducer of choice (vitamin D, glucocorticoid, etc.). Reporter gene activity is then measured. Test compounds are added to the activated cells, and compounds are identified that eliminate/diminish the reporter gene activity. Alternatively, inhibitors that lower reporter gene activity in the absence of an inducer may be identified; the presence of inducers may bias the type of inhibitor identified in such a screen.

Those skilled in the art will recognize, or be able to ascertain using no more then routine experimentation, numerous equivalents to the specific polypeptides, nucleic acids, methods, assays and reagents described herein. Such equivalents are considered to be within the scope of this invention and encompassed by the following claims.

What is claimed is:

1. An isolated nucleic acid comprising a polynucleotide sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO: 10, or a polynucleotide sequence having substantial sequence homology thereto that is capable of conferring transcriptional responsiveness on an operatively linked RANKL gene promoter.

2. The isolated nucleic acid according to claim 1 further comprising a promoter that is transcriptionally responsive to said polynucleotide.

3. The isolated nucleic acid according to claim 2 wherein the promoter is a RANKL gene promoter.

4. The isolated nucleic acid according to claim 2 wherein the promoter is a Herpes simplex virus thymidine kinase promoter.

5. The isolated nucleic acid according to claim 2 wherein said nucleic acid further comprises a reporter gene operatively linked for transcription by the promoter.

6. The isolated nucleic acid according to claim 5 wherein said reporter gene encodes luciferase, chloramphenicol, acetyl transferase, beta-lactamase, green fluorescent protein, or beta-galactosidase.

7. A host cell comprising an isolated nucleic acid having a polynucleotide sequence set forth in SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, or a polynucleotide sequence having substantial sequence homology thereto that is capable of conferring transcriptional responsiveness on an operatively linked RANKL gene promoter.

8. The host cell according to claim 7 wherein the host cell is a mammalian, avian or insect cell.

9. The host cell according to claim 7 wherein said isolated nucleic acid further comprises a promoter that is transcriptionally responsive to said polynucleotide sequence.

10. The host cell according to claim 9 wherein said nucleic acid further comprises a reporter gene operatively linked for transcription by the promoter.

11. The host cell according to claim 7 wherein said host cell endogenously-expresses a steroid/thyroid hormone receptor or transcription factor capable of interacting with the polynucleotide sequence and regulating RANKL gene expression.

12. The host cell according to claim 7 wherein said host cell endogenously-expresses the vitamin D receptor.

13. A method for identifying a chemical entity capable of altering RANKL gene transcriptional activity, comprising:
(a) providing a host cell according to claim 10;
(b) exposing the host cell to a chemical entity; and
(c) measuring and comparing reporter gene expression to that of a control cell that is not exposed to the chemical entity wherein a higher or lower expression level than that of a control cell indicates that the chemical entity is capable of altering RANKL gene transcriptional activity.

14. The method according to claim 13 wherein said host cell endogenously-expresses a steroid/thyroid hormone receptor or transcription factor capable of interacting with the polynucleotide sequence and regulating RANKL gene expression.

15. A method for identifying a chemical entity having reduced hypercalcemic activity, comprising:
(a) providing a host cell according to claim 10;
(b) exposing the cell to a chemical entity; and
(c) measuring and comparing reporter gene expression to that of a control cell treated with a known hypercalcemic agent wherein a lower expression level than that of the control cell indicates that the chemical entity possesses reduced hypercalcemic activity.

16. The method according to claim 15 wherein said host cell endogenously-expresses a steroid/thyroid hormone receptor or transcription factor capable of interacting with the polynucleotide sequence and regulating RANKL gene expression.

17. The method according to claim 15 wherein said known hypercalcemic agent is 1,25(OH)₂D₃.

18. The method according to claim 15 wherein said chemical entity is a vitamin D analog.

19. A method for the controlled expression of a gene, comprising:
(a) providing a host cell according to claim 9 further containing a gene operatively linked for transcription to the promoter; and
(b) culturing the host cell under conditions to express said gene.

20. The method according to claim 19 wherein said host cell endogenously-expresses a steroid/thyroid hormone receptor or transcription factor capable of interacting with the polynucleotide sequence and regulating RANKL gene expression.


SEQUENCE LISTING (10 sequences)

SEQ ID NO: 1 - DNA, 1200 bp, Mus musculus
cagtctaagggtttatcaagggagaaagaagcagggtcggcacaacttgatgtttgccatgcttgggcctgttagcccttcgataaataacatgtatttatttatttatgaccatgataaattggcaccagagttatatcccaacacttagtatctgtaggcctagggtcctcaacaatttccacccatagaagagagatgggcaacatcacccagtaaacatcgccacgcaatgagatcatcacatgagttcccaagtgaacctactcagttgcagccagatcagacctgctctcctaagtggaagagaaagccaagtcctgggtgaggctagacaggctgacactatgaagctggacagtttggctgtggtggagccattaattatagtgtgtcctgatcatgtctatttgtttaaactctgcttggtgttacaaaaacacccccgttcgacagtaagcacccaaatgtgagtgttcctgcagctatattcatcttaccatgactgtggctacaggtctatggtgagaacactcagccagataaacaggaagatagggggtggcatgccatgtcccattgggactgaaccctgaaagatgtgggctcagctaggtcccagagaaagtgaccttcttgacacagttgtggggtaagttgagtggtccccaggacaaattggcatctcttatatgaccagttagacatttaacaggttaaactggtaaactggcagataaagagatgaactctaaagaggtaaactttcagctctttcctaactgtcttttgttccggccccagaatgtggcaaagtagagcagagagcagcttgtttgtttaagatgttgactgacctctgagctcatgtatgagctgattcacaaagttcttaaagttgaattatcctagtacttctggaccagtggaaatgaactatgcagccttcttctgtgttttagaaaagaagtgaatgggattgctccctctcaactttgtcaagaattctaccctcttctgttttcctccacagtcactgggtcacactgagcctcaacgaatttcaaaacagtgttttacacgcagaatactagtgaacagaaggagatttgacatgattttaaagacttacttttatttgaaattttgtgtctatgtgtacggctctgtgtgggtatatgcatgtgtaaatgtgggatc

SEQ ID NO: 2 - DNA, 1600 bp, Mus musculus
gactcacgactaaatccgtaagagtgtgctaacaagcttggaatgtggggttggatttagtatgagctgtttgctactcactctgttctcttcactgaagtggggaatggcagtgagcactgagggctgtgttataaacttgcttttcaatgcttccccatcaacttttctttttgctttcctttctcagagcacactggaaagtataagcaagcacacaagtgttggatctgtatgtttagttcggattcaaccccattgttatttcctggagtccttccaagagatcttacaagttatcaatctcgccacgacagagtgctttctgaccacttcccctttgcaattaaggactgtgaccactcgcttacgcacagactgagaacagcagccataaccccaaaggaggtcagaagctcccagctctccccaccaagggggcttagccgagaacctgataccttgtgttttccagctgtggctaaggctctgaccataggaactgggaacacccaccaacacccgtctcccccaaccccacttccttggcttcaaatgccattaaacatgtttgcagtgcgtttgtgaatgttttgagtggagtttggtttctggtgtttattgttgaattgctaaagatttggagaggaaacagacagacagacaggcagacaaatagacacaaatctctggtaagatttgcacatgagcagatgtgctcagtatgaattgggtggccatgagctctgtgcatttctgggctctctctcactagagccccagaatttggtttgctggtctacaaataaaacttactctagacacagagtaaggagccctttatttgcataggaagcctgccagcctggcctggaagatgataggtcacagccagcgtggtacaggatatttctattcactcactcctttcaaggatgtgtctaatgtgggggctgagcagttgggcagggccatctcttcccctgttgccagttctactgtttcacttaaatccagtactgtgcctctggttgcttgcctcgtcagctcttctcagcttaaagtttgaagaggcaagataagctcacccagactcatcttgggaaactgtgggaggcttgggagtggggctggctgggttcatgatggaaaggtcagattgcactgtgcggtccgcaggctttcccagctgggcaggaaggctttccccatctgacttcatcatggtacgggttgcgctcacccacataaactccacgggggcggaggctgtgcctctgttgccactgtgtaagtgtgtagtcacctttatctcagcagcaggtgagcatcacactgtaggtgccactttgttgaatgagtaaatgtgtataaatagacccaatgcatttcatttgcaggggtcttctttaagatacaataagttccaggtctttattcataggtgtgtgcttgccagaatattcatattagttttaattttagccaatgtgtctattgtatctgctgttcttttccttgacagcatggcatttttgctacctggttgcactatacagagtattttatggaaccaacatttttactagcacagatccct

SEQ ID NO: 3 - DNA, 900 bp, Mus musculus
gataatttatgataacacataaggtcaaaagagctcacttactaaacataacccaagactacagtacagtcataataacactgagtttttattcagtactacatttcccaggtgctacattgtgttggacctggctaggactatctgataaacttacagtaggtaaaagtccattttcatagctgaatgtccatttggaaatcatagctgtcatttggaaatgatgttgttgaacacatcctcaaggtcatggtcagaccacccctggtctccttcctcttttcagacctatgaatgatgctctttgtaacagggactatttttttttttttttgtgtgtgtgtgtgtgtgtacaacaataagatatttctgagaaggatcttggagtcagatttttctttatgtagtggtatcttgtgttgtttactttagaaatagtttctagtaggaaaaaaagagagagagagctgtgtcctagaagaattgtccccaagagcccatgaacagggataatggtttgcaccaccgctttctggaaagcaaatgggattttgacatgaactcggaaaggggtcagaccacactgacatatattttctggtttctaatcttactttcgctaccactgtggactgcattccataaccctctaacctagcgtccagccattctaccgctttttatcaactcccagaatggtctcagcacagcatgtctcttgctgagggcagggggaccagtgggtggccagacctcaatctctgggcagcaacacccagacttctactctactctgaaaaaagcattatgacatcaccgcgggccaatcaaatcagtcgtctcggatggctattcctagatgattttgtctatgatactgtctcgtagcctaaataactg

SEQ ID NO: 4 - DNA, 900 bp, Mus musculus
tgtttgttgaagaaaaataattgcagtgaaaatgagaaaaatatgtgctgtgaagcaaaaattcctatagcctctcctgacctgtgtgttcccgaaggaagcataatgtctgtatttatttttgagatgaaatttctcattactgtcagtgagactcagtcattcctgaggttcttcggagacaattcagctttttgttttccccagatgaaagatttattgatggcacacaggggcagaaagccaaggcaaatgatacatggatgtaaaatagacaaggcctgcacatcacttctggtgtctagggtctcactgcagtgtgtcgctgtgatactgtgttgttggttcgttgtcctccctggtcaatacagcctcgttacttctgaggcccatttccaaaagcaacaactgagtctcccaggagaaccacggggcagctcaactgagtgacgggactttgagatcgtctgaatgagaaccaagtttgaagataatgatggggttgacatttgtgtcacctggaacactcactgctgacatacttccctcacatttgtagtgctaatgtaggggtctgctttcagcaaggatgaacaaatttgttttgcatggtcccatggtgtgattctatgatatctggcattttggcatttgcaagatggatgactctctgtgccttgtggctgactatgaggtaaaccaagaattatgtatctctagtgcattctttcccatgagtgcatacttttggattgcgagcccagggaaatgaagtagatggggggggaggcttttttcccttaaagattcctcattattcaggtaatatgaatgtgggacaagcatgtttgtaatttgagtctacaatgcctgtggtcaccttaatcaagtgtatggtga

SEQ ID NO: 5 - DNA, 1900 bp, Mus musculus
gtcatttgaagccataatggcagctgcttaatgaactattggactgcctaaatcacaggatttaagtctgaagagtaacacagagaagggactatttataaataatctttcttgaacagctgggtggaatttgggtccattcattggatccatgttcagcatttccaaagattaaaaatgtgaatcataccaatgaaactggcattggctgacttcagaatatcaccacatcaaacacccagttggcgtcagggaaggaagtttgatgtcactgtatgttgcctatatgttttcttaacccaacagcaagcttcctgtggaacccctagagctgatggaatgtcttcatagatgacacataggttgtcccaacactcctaccatctttttatacttttgagcaaagtcatccagccaaaagcccatttggaaaataaattctgagttatatacatgagaggacaagagaaaaagacttacaaatgacctaaggtagaaatcatatagaagatactgagctggggaaaagctgaaatatgatacatttaccaaaataaatctcacttcgggtcactattatagttacacggtttaccaatcagccaacttaccatttagatttttttcctcttctttttgtcaggagtgttatttacacatgttattcttcttcagctaaaactttctctgcttaactaactctggcctatcttgatacttttccaaaagaaacagagagaaacacactagcagaaagtccaaaggcaccccaggagtcaaaggcagctttgggaacctttctggactctcaggtctctgagttcttcagagaccatagaaatcaacaaacgtaggaacaactctattctctagaaccaatattttctgtggggaaaaaataaccaaaatttaccaaaagattctgatagaaactgtcttcatgcttcccctagtggctgggttctcatccagaaatggcccagacccagtgtctggtgtcttaacaagggtcaagaggggcctgacttctgtttttgatgttttgtccacgtgcctgttaaaatggggagggaggtgcatttgattctggatttgttgactcttgtgcttgggttctggagctctttgtgtcctgtccctcccagggacagagaccgggacatatcctggaaacggtctggcagttcactgtccttgagttctctgtgggtgaactcaggcaaccaacaactcggtgactctggcagctgtttttgtttcccagtcctgggcaccttgccctgcagccttgtacacggagggtgatgcctggcatcctgcccagtgccaccaggaatgagtgcagccccagggagtactgccaagtcatgttggcaagctgctttctggccactggatgaatgcattggtctgagccctgagaacagtgtgacaggaacatcgggaagacagcacagagtgctggatgcagctgtgcatcctgactccatcttctctttgtttacacactgctctcgcctcattgtgcaattgtggtgactgaggcggctgttctctggatagaaaccacgggcttcactgtcctccctgtctgtagtagcagcagcagcaaaatattagtgggaaatcatgctgcccgaatgggttctggttgaagtcaaaagactgaagtcacctcaacgataaataagaaagcaactcttgccagtcaacacagattttgtagacccgctgttggtaagaatgtggtgcagtgaagtctgaagtgtgaaaagttgccacactcctcatttgccatttgagccccttaaaggctttcaacacactcctcagccttgcttttcctatttcaaagctttgcttcatatggaacatttttcaaatgtaaaagaacaaaaag

SEQ ID NO: 6 - DNA, 1062 bp, Homo sapiens
ttcagcccattgacaaagcccatttatttcagcctatggtaaactctgacataggagtatctcctaatctttagtgcctgtaggtctgaggtcctcaatccttttcaccagtaggacagcagtggtaaaattcagccagcaaacattgcatgcaagacttcacttttatagcaagtactgaatgaaccatgtactcaatgccagcctagctggacctgctgtccttctaaatggagcaattgcagaaaaagcaaagttctatggtgaatccaaacagtctgaaaccatgaagctgggcagtctggctgtgtggtgcagcccattcattatggtgtgtcctgatcatgtttatttgtctaaactcttcttggtgttgccaaactgccctccttggacattaaacaacagaagagtgagaagccatgcagttgtagtcatctgtggctgcaggtctatggtgaggaggcccagccagactaatagggagatctgggacacagggagccatgctgtgtctcaccgggatggggccctacaagccccaggttccatggggcacagttttggtgtgactggtggggtccctagagccaacggccatctcctttatcatcagccaaaagttgttcaggttaaactgatggactggggggagggagatgggtgatgaggagagggaaacgctctcaagtcctttcctaaatgcctttttcctgccccagactgtggtaaaatagagcagagaccagctggccagccttgtttgtttaagttgttgactaatctctgagttcatgtgctgagctggttcatgaagttcttgaacttttattatccttggacttctgacacaatgagaatgaaataagaaactgctactatatttaaaagcagcctataagattgagtgacttaaatgtttgactttgaatccaaagattcaaggaattaaaaaaattgtataatattcactgagagctactctgtgccaggcacttatttgaaatataatgttttaaatgcaaaatgctaataagcaaatgtaaataagagattctagaa

SEQ ID NO: 7 - DNA, 1324 bp, Homo sapiens
gtgatctcttcactcaaaaaaggaaatggcagagaattctattggctttgttgtgaactctacctctaattcctttccctctgcttcctttctttttcttttcagaacatgttggaaattataagcaaacttgtaagtgatgtatgtatatttagtttcatagacaacctcagtgttactttctggcattcttccaaagtatcttacaagttatcaatcttgccaggacagagtgtgttttctgaccacttcccctttgcaattaaggactgtgaccactcgcttacgcacagactgagaccagcagccttaaccccaaaggaggtcagaaactcccagctctccccgccaagggggctagctgagaacccgatactgtgtgtttcaaggctgtggctaaggctgtgaccataggaactgggaacacccaccaacaccctgtttcccccagccccacttccttcgcttcaatgccattaagcatggttgcagcgcctttgtgaatgttttgagtagagttaggtttctggtgtttattgttgaattgctaaagatttggagagaagacaaacagacaaacagacatacaatgaagcagatgacaaacacccctggacagactagcactatgagcagacacgctcaggacgacccagggtcctgcggagaggtccgggaaggtctgggctctcatcagagccccaggatttggtcagttgctctgcaaataaaacctgccttagacatccagcaaagaggctttatttgcataggagatttgccagtctggcctccaagatagtaggtcacagtgagggtggtggtgagttttcctgtttactgtctacctttgtggacacattgaatgtgggggctgaaaggggtcagcaagggctgtctcctctcctgttttcagatttggtgtctcagtgaaatccagtgctttgcctctttgattgtgctctgtctcagtgcttctcaatagcatgtgatgtgcagagaagtaaaagaaatgaacaaagatttctcttctgaaggtgtgggagggcatgggagtggggctggttgggctgatgatggaaaggtcagattgcattgtgtcgtccacaggctttccagctgggcaggaaggcttccccctctgactgcatcatggcacttcttgtacatactcacttacccacagaaactaaactccgcatggatagaggctgtgcctatgcggttggctattgcacacccagcatcaccacagtgcctggcactatcagactcttcgtgcctatttgttgaaatgtctacaaatagactaaatacatatcag

SEQ ID NO: 8 - DNA, 725 bp, Homo sapiens
gcaataggcaaaaatctatcttcatagctgaaatctgttttggaattcattgctgcctttctggaaagacactgtttaattctgtctcctgggtgtgtgaccctatggtctctaccttctttgtagatctgtgaataaagctctttgtaatgaggccactttttgtgcaacaacaacaaaatgtttctgagaaggatgttagagccagactttctttaagtagtggtgtctggtgttgtttacttttaaaatagtttctagtgcaagggagaattgtgtcctagaagaactgtcccaaggagctcatgaacaggaataatggtttgtaccactactttctggaaagcaaacggggattttgatgtgaacccagaaaggggtcggaccacattgacatatattttctggtttctaatcttacctccactgccattatgcactgcattacacaaccctctaccctaaggcccagccaccctacagccttttatcaactcacagaatggtctcagcacagcatgaatctagatgagggcttagggacccgtgggtggccagacctcaggctctgggcatcaacaggcagacctccagttctgccctgaaatagaattatgatatcattgtgagccaatcaaattgcctgtctgagttcagtcctggatgctctggggtggtaattcccagatgatttgatctatggtattttctagtagtctaatttg

SEQ ID NO: 9 - DNA, 983 bp, Homo sapiens
tgtttgctgaagagaaataattgtggtgaaaatgagaaaattatgtgctgtgaagcaaaattctatgacatatcctgaccttatgtatcccctcaggaagcataatggaaatatttatgtttgaaataaaatttctcatgtacttccagctacactcagtgagtcattcctgaagatccttggggaaaatttagactatgtttcttggatgaagattgattggtggcagacaggggcagaacatcaagggagatgatatatatgtataaagtggaggaaactagactattacttctgctttttcaagtcaagatgtgatctgctgtcataatattatattgctggcttggcttttttttgtttttttctgtcagtgtggttattgttacttcatctgtggcctgtttccaaaagcaatgagtgagcccccgcaagataatcactagattatctggttctttcatgactaataggccaggggaatgtactaaacaaacagttcagctcgaacgtgttgcaggggattgaaactgtctgaatgaaaataaaatatgataaaataatataattcacatgattcatcttctgcaggacaaccaagtctgacattcctattccatataagaagtggtaatttcacagtgtacattcagtaaggatataaaatgtaattttggcaggtttcatggtataaagttataatatggttttgggggtttataatatggatgactttatttaccttgtggctgactatgaggtaaacttgcaaatatgtgtccatattacattcatctactatgagcaaatactttcgggttgtgaacccaagggaagaaagtggtaaaatggcttattttgccgcatgaatttcagtttactcgggtaatacgaacgtggcaggggcacatttgtaatttagggagttataacatcagtggttacatttaattaaatgaccatcaacatgggattatttagga

SEQ ID NO: 10 - DNA, 3495 bp, Homo sapiens
gcaatttgcaagttgtgactgtagttgcttgatgaactattgaactaattgcctacattgctgaactaactaaagaatttctttaagttagaggatgatgacagggaagagactatttacaaatagtgcttcttgtacagctgggtagaatttagggtcattcatttatgttcagcatttccaaaggttaacaatatgaatcatacaactgaaattggcatcacatggctttagaatgtcaccacatcaaacacgcagctggcgtcagggaaggaggcctgatgtcattgtatgtttggcctatatgttttcttaacccaacagctagcttcctgtgaaacagtacatccgcatggaatgtctccgtggactacacgtagagttcaggatacttctgccatcttagttgtgtgagtgagccaaatcacacagccaaatgcccatttggaaaataggttctgaatttatacacatgaaaaaccaagggaaaaagatttgcctatagagagatcacaccaaggatactgagctggggatgattaaaataaaagtgacattgaccaggtaaaattccattttaggtgactgtcgctgtcacacagttctagtgatacatttgcagcaaccaactttgcaagctacaaaggatcagccttatgatccagatttggcctttgatttgggggatgctatttgcattatggcactcttcctgtgatgagctttatgacttcttattacccagtacataaaactctgctattgcattgtgatgactattcattttaatcttgaagaatttataacctgactcgaggtgagtgatttaaaatagaaaaacaaccaaagacatatgttacaaataccagtgcttcccctgagttctctgaggctagtgttgggctctcttgaggccagcatggtcatggtttctgctctaccacctctgtgcatcatgaacctggagtgtctataccttccccatgtttagaacggacagatttgatggatgaagagtctccagctaaagtttcctgagggaaggttctgtctcttatgcagcattatataaatctagtgtctggcatggtgcctaatgtatagtgggaatataagtattttgattgaatgaataaacaaatatagatgtgcacatgggaagtagaccacacaggaaaagttactgtgtattgattcatttgctgactagcaaataggatgtttggaaataaattggtcatattgctattaccctccaaataatcacattatttctacagattgcaatataggcataagcatcatgtgaagtaggtatccattctgattataaaaataaggtggtgccatctgggaagtgaggagcgcctctgcccggccgccccatctgggaggtgaggagcgtctccgcccggccgccccgtctgggaggtggggagcgcctccgcccggccgccccgtctgggaggtggggagcgcctccgcccggccgccccgtctgggaggtggggagcgcctccgcccggccgccccgtctgggaggtgtacccaacagctccgaagagacagcgaccatcgagaatgggccatgatgacgatgggggttttgttgaaaagaaaagggggaaatgtggggaaaagaaagagagatcagattgttactgtgtctgtgtagaaagaagtagacataggagactccattttgttctgtactaagaaaaattcttctgccttgggatgctgttaatctataaccttacccccaaccccatgctctctgaaacatgtgctgtgtccactcagggttaaatggattaagggcggtgcaagatgtgctttgttaaacagatgcttgaaggcagcatactggttaagagtcatcaccactccctaatctcaagtacccagggacacaaacactgcggaaggccaaagggtcctctgcctaggaaaaccagagacctttgttcacgtgtttatctgctgaccttctctccactattatcctatgactctgccacatccccctctctgagaaacacccaagaatgatcaataaatactaaaaaaaaataataataaaaataaaaaaaaataaggtggtgcatcgaatggcaaagtgattaggaatccctttggacagttcattcgtaaggaatttgattctgaactttaaattccttctggtaaggtgagctagcttgctttagctaacacttgctctgcctgattcttgcatatcttgatgcctctgttataaagcacagtagtgactaaatcagtagaaaacactgcagccactccatgggtcagggggctggaggggatgatatttaaggacccttctgtccctgaattgttctaagcctattaaaaaaccaacaaaataagacaacaaaaaaggaaaaagcccacttccagaagcagtcttttctgtgaaaaaaaaaaaaaaaaaattaatcaatctattatttggcatcttgttgaaaaggcacagttaaaaaaaaagattatgagacaagtttttttaatttgcatttctacaagttgctttgctgtcatctagaaatggctcaagccttatgtttcggatcttagcatgagtcaggaggggcttagcttctgcccccaacccttatccacctgctgatggtgatggggaggcttttgcatttgtttccatgtcagctgacccttgggcttgggttctggagcttgcaatgtccctcacagggacagggactgatcctggaattggcctggcagtgtactcacccagcggggtgaactcagacaaccgacaacttggtgacttcgacagatgtttttgtctcaaagccttgggcaccttgccctgctgtctcgcatgcagagggtgatgcctggcgccctgcctggtgccaccaggaatgagtgtagccccagggagtactgccaagtcatgtcggtgagctgctttctgggggccaactggccactggataaatgtgttggtcggcgtgttcagaacagtcaggggtcacactagcttaagtatctcactgctttatgtggctgtaacttctaagtctatgttctgtgtttcaagccattttgtttatacattgcatgtgcttcattgtgcaattgtggtgactgagggggttcattggacagaagccataggtttcacggttgtgtctgtagcaaaatattagtgggaaaacatgctgccccgagtgtattctggttcaactctgaagattgaagtcacctaaacaataaataagagataaaccctgccagtcaacaacagattacatagacccacttttggtaaagcatggtgcagttgaaaagcactgagcttgtagtcaggaagcctctgcatattctgcttcaccatttataagctgggcccttgaatgttttcaatgtattttaaatttggtcctgttcctattttaataactttattttgtattgaagtatctctcaaattaaaaagaaaatgag